(19) United States
(12) Patent Application Publication
(10) Pub. No.: US 2005/0069508 A1
(43) Pub. Date: Mar. 31, 2005

(54) Cosmetic composition comprising a dispersion of at least one wax in at least one volatile oil

(76) Inventors: Karl Pays, Saint-Maurice (FR); Therese Daubige, Beaumont-eu-Veron (FR); Patrice Styczen, Gif Sur Yvette (FR); Yohann Bichon, Maisons-Alfort (FR)

Correspondence Address: Thomas L. Irving;FINNEGAN, HENDERSON, FARABOW,, GARRETT & DUNNER, L.L.P., 1300 I Street, N.W., Washington, DC, 20005-3315, US

(21) Appl. No.: 10/912,702

(22) Filed: Aug. 6, 2004

Related U.S. Application Data
(60) Provisional application No. 60/495,895, filed on Aug. 19, 2003.

(30) Foreign Application Priority Data
Aug. 6, 2003 (FR) 03 09710

(51) Int. Cl. A61K 7/021; A61K 7/025

(52) U.S. Cl. 424/63; 424/64

(57) ABSTRACT

Disclosed herein is a cosmetic composition, comprising: (i) at least 20% by weight of at least one wax having a starting melting point of greater than 45° C., relative to the total weight of the composition, (ii) at least one dyestuff, and (iii) at least one volatile oil, wherein the at least one wax is dispersed in the at least one volatile oil. Further disclosed are methods for preparation of the composition and methods for caring for and/or for making-up the skin and/or the lips, and for making up keratin fibers.

This application claims benefit of U.S. Provisional Application No. 60/495,895, filed Aug. 19, 2003.

Disclosed herein is a cosmetic composition comprising a dispersion of at least one wax in at least one volatile oil. Further disclosed herein are care and/or make-up compositions for the skin and/or the lips, and also make-up compositions for other keratin fibers, such as the eyelashes.

In general, non-rigid cosmetic compositions comprising waxes comprise at least one wax or a mixture of waxes dispersed in a liquid phase. It is mainly by means of the amount of wax and of the other non-volatile ingredients, reflected by the solids content of the composition, that the desired application specificities for the compositions may be adjusted, for instance their fluidity, their covering power, and, in the case of make-up compositions for keratin fibers, their curling power, and also their thickening power (also called charging or making-up power).

In practice, there are essentially two types of formulations of compositions comprising waxes, i.e., firstly, aqueous compositions in the form of an emulsion of at least one wax in water and, secondly, anhydrous compositions or compositions with a low water and/or water-soluble solvent content, known as “waterproof compositions”, formulated in the form of a dispersion of at least one wax in nonaqueous solvents.

The present disclosure relates to compositions in the form of a dispersion of at least one wax in at least one nonaqueous solvent, for example, at least one volatile oil.

Waxes are compounds that may be cosmetically advantageous. They may provide the compositions comprising them with certain properties such as consistency and smoothness, but also give the film applied a notable staying power. In the case of make-up compositions for keratin fibers, waxes may be capable of improving the curling power and the thickening power thereof.

Conventionally, compositions in the form of a dispersion of at least one wax in at least one nonaqueous solvent have a wax content which does not exceed 20% by weight.

Now, when seeking to increase the wax content, one is confronted with a problem of lack of fluidity. The composition may become too thick on application and, in addition, may no longer have the shapeability required for it to be applied homogeneously, or may no longer be taken up using the usual accessories, such as brushes.

One aim of the current disclosure is to overcome at least some of these drawbacks and to propose a cosmetic composition which has a high wax content allied with optimal cosmetic qualities throughout its storage, inter alia a satisfactory and virtually constant viscosity over time, whatever the temperature or the changes in temperature it may undergo.

For example, one embodiment disclosed herein is a cosmetic composition comprising:
(i) at least 20% by weight of at least one wax having a starting melting point of between 45° C. and 100° C., relative to the total weight of the composition,
(ii) at least one dyestuff, and
(iii) at least one volatile oil.

Another embodiment disclosed herein is a make-up composition for keratin fibers, comprising:
(i) 20% or less by weight of water and/or of at least one water-soluble solvent,
(ii) at least 20% by weight, relative to the total weight of the composition, of at least one wax having a starting melting point greater than 45° C.,
(iii) at least one dyestuff, and
(iv) at least one volatile oil.

Further disclosed herein is a process for preparing a composition as defined above, comprising the continuous blending of at least one wax in accordance with the invention, going from a temperature above the melting temperature of said wax to ambient temperature by continuous cooling.

A further embodiment disclosed herein is a method for preparing a composition as defined above, comprising dispersing at least one wax in the form of particles having a size ranging from 0.5 μm to 30 μm, for example ranging from 1 μm to 20 μm, in at least one volatile oil, said at least one volatile oil being at a temperature below the melting temperature of said wax in the form of particles.

Additionally disclosed herein is a cosmetic method for caring for and/or for making-up the skin and/or the lips, in which a composition as defined above or as obtained by one of the processes as defined above is applied to the skin and/or the lips.

Also disclosed herein is a cosmetic method for making-up keratin fibers, in which a composition as defined above or as obtained by one of the processes as defined above is applied to said keratin fibers, such as the eyelashes.

Another embodiment disclosed herein is a cosmetic method for making-up keratin fibers, comprising applying to said keratin fibers, such as the eyelashes, a composition comprising (i) 20% or less by weight of water and/or of at least one water-soluble solvent, (ii) at least 20% by weight, relative to the total weight of the composition, of at least one wax having a starting melting point greater than 45° C., and (iii) at least one volatile oil.

In certain embodiments, the compositions disclosed herein may exhibit a higher rate of drying than the conventional compositions based on the dispersion of waxes in a nonaqueous solvent. This may make it possible to reduce the time required for implementing the processes using such compositions, and for example in the case of a make-up process, it may reduce the risk of transfer of the make-up onto an adjacent surface. This also may make it possible, where appropriate, to apply several layers of said composition in a satisfactory amount of time and to thus further reinforce the effect obtained with these compositions, such as the make-up effect thus obtained.

As used herein, the expression:
“between . . . and . . . ”, is intended to denote a range of values, the mentioned limits of which are excluded, and
“from . . . to . . . ”, “ranging from . . . to . . . ” and “varying from . . . to . . . ”, are intended to denote a range of values, the limits of which are included.
   At Least One Wax

The at least one wax as disclosed herein may be a lipophilic compound that is solid at ambient temperature (25° C.), with a solid/liquid reversible change of state, having a melting point of greater than or equal to 30° C., for example, up to 200° C., such as up to 120° C.

By bringing the at least one wax to the liquid form (melting), it may be possible to make it miscible with oils and to form a microscopically uniform mixture, but on cooling the mixture to ambient temperature, recrystallization of the at least one wax in the oils of the mixture may be obtained.

The compositions disclosed herein may be characterized by the presence of at least one wax having a starting melting point of greater than 45° C. The starting melting point of the compound, hereinafter referred to by the abbreviation “mpₛₜₐᵣₜ.”, corresponds to the temperature measured when 5% of the melting enthalpy has been consumed.

According to one embodiment, this starting melting point may be greater than or equal to 50° C., such as greater than or equal to 55° C., or greater than or equal to 60° C.

In the context of the present disclosure, the melting temperature corresponds to the temperature of the most endothermic peak observed by thermal analysis (DSC) as described in ISO standard 11357-3; 1999. The melting point of the wax can be measured using a differential scanning calorimeter (DSC), for example the calorimeter sold under the name MDSC 2920 by the company TA Instruments.

The measuring protocol is as follows:

A sample of 5 mg of wax placed in a crucible is subjected to a first temperature rise ranging from −20° C. to 100° C., at a heating rate of 10° C./minute. The sample is then cooled from 100° C. to −20° C. at a cooling rate of 10° C./minute, and it is finally subjected to a second temperature rise ranging from −20° C. to 100° C. at a heating rate of 5° C./minute. During the second temperature rise, the variation in the difference in power absorbed by an empty crucible and by the crucible containing the sample of wax is measured as a function of the temperature. The melting point of the compound is the temperature value corresponding to the top of the peak of the curve representing the variation in the difference in power absorbed as a function of the temperature.

The starting melting point of the wax can also be measured using a differential scanning calorimeter (DSC), for example using the calorimeter sold under the name MDSC2920 by the company TA Instruments according to the same protocol as that described above.

As waxes with a starting melting point of greater than 45° C. but less than 50° C., mention may be made of montan wax (mp_{start.}=47.9° C.), ozokerite (mp_{start.}=46.3° C.), and the wax obtained by catalytic hydrogenation of olive oil esterified with stearyl alcohol, sold under the name Phytowax Olive 18L57® by the company Sophim (mp_{start.}=47.4° C.).

As waxes with a melting point of greater than or equal to 50° C. and less than 55° C., mention may be made of rice bran wax (mp_{start.}=51.4° C.), candelilla wax (mp_{start.}=50° C.), and ouricury wax (mp_{start.}=51.4° C.).

As waxes with a starting melting point of greater than or equal to 55° C., such as greater than or equal to 60° C., mention may be made of camauba wax (mp_{start.}=63.5° C.), the waxes obtained by Fisher-Tropsch synthesis (mp_{start.}=60.5° C.), certain polyethylene waxes such as those sold under the name Performalene 655® by the company New Phase Technologies, Polyethylene AC 540® by the company Honeywell, Polywax 2000 T-6® by the company Petrolite (mp_{start.}=125° C.), PED 191 and Epolene N-14® by the company Eastman Kodak (mp_{start.}=120° C. and 106° C., respectively), and certain microcrystalline waxes such as those sold under the names Tisco Wax 88® by the company Tisco and Microwax HW® by the company Paramelt. Mention may also be made of waxes obtained by catalytic hydrogenation of animal or plant oils having linear or branched C₈-C₃₂ fatty chains, such as hydrogenated jojoba oil (mp_{start.}=63.2° C.) and bis(1,1,1-trimethylolpropane)tetrabehenate sold under the name Hest 2T-4B® by the company Heterene (mp_{start.}=61.8° C.).

According to certain embodiments, use may also be made of waxes provided in the form of small particles having a size ranging from 0.5 to 30 micrometers, such as from 1 to 20 micrometers, or from 5 to 10 micrometers, referred to herein as “microwaxes”. For the purpose of distinction, the waxes used herein in the form of larger-sized fragments are referred to as “conventional-type waxes”.

As microwaxes having starting melting points of greater than 45° C. which can be used in the compositions disclosed herein, mention may be made of carnauba microwaxes such as that sold under the name MicroCare 350® by the company Micro Powders, microwaxes of synthetic wax such as that sold under the name MicroEase 114S® by the company Micro Powders, and microwaxes comprising a mixture of carnauba wax and of synthetic wax, such as that sold under the name Micro Care 325° by the company Micro Powders.

Among the waxes mentioned above, the polyethylene waxes such as the wax Polywax 2000T-6 from Petrolite (mp_{start.}=125° C.), the wax PED 191 from Hoechst (mp_{start.}=120° C.), and the wax Epolene N-14 from Eastman Kodak (mp_{start.}=106° C.) have a starting melting point of at least 100° C.

In the compositions disclosed herein, the at least one wax having a starting melting point of greater than 45° C. may be present in an amount ranging from 20 to 70%, such as from 22 to 65%, or from 25 to 60%, by weight relative to the total weight of the composition.

The compositions disclosed herein may also comprise at least one additional wax having a starting melting point of less than or equal to 45° C.

Such waxes may be chosen from at least one of waxes with animal, plant, mineral, and synthetic origin.

The waxes which may be used in the compositions disclosed herein may have a hardness ranging from 0.01 MPa to 15 MPa, or a hardness greater than 0.05 MPa, such as a hardness greater than 0.1 MPa.

The hardness may be determined by measuring the compressive strength, measured at 20° C. using a texturometer sold under the name TA-TX2i by the company Rheo, equipped with a stainless steel cylindrical moving body that is 2 millimeters in diameter, and measuring the change in strength (compressive strength or stretching strength) (S) as a function of time, during the following operation:

The moving body is moved at a rate of 0.1 mm/s and then penetrates into the wax to a penetration depth of 0.3 mm. When the moving body has penetrated into the wax to a depth of 0.3 mm, it is kept fixed at this point for 1 second (corresponding to the relaxation time) and is then withdrawn at a rate of 0.1 mm/s. During the relaxation time, the strength (compressive strength) decreases to zero and then, during the withdrawal of the moving body, the strength (stretching strength) becomes negative and then again increases towards zero. The hardness corresponds to the maximum compressive strength measured between the surface of the moving body and the wax at the time they are brought into contact. The value of this strength is expressed in MPa.

To measure the hardness, the wax is melted at a temperature equal to the melting point of the wax +20° C. The molten wax is poured into a container that is 30 mm in diameter and 20 mm deep. The wax is recrystallized at ambient temperature (25° C.) for 24 hours and is then stored for at least 1 hour at 20° C. before the hardness is measured.

By way of illustration of the waxes which may be suitable for use in the compositions disclosed herein, mention may be made of hydrocarbon-based waxes such as beeswax, lanolin wax, Chinese insect waxes, sumac wax, paraffins, certain polyethylene waxes and waxy copolymers, and esters thereof.

Mention may also be made of waxes obtained by catalytic hydrogenation of animal or plant oils having linear or branched C₈-C₃₂ fatty chains. Among these, mention may be made of isomerized jojoba oil such as the trans-isomerized partially hydrogenated jojoba oil manufactured and sold by the company Desert Whale under the trade name Iso-Jojoba-50®, hydrogenated sunflower oil, hydrogenated castor oil, hydrogenated coconut oil, hydrogenated lanolin oil, and bis(1,1,1-trimethylolpropane) tetrastearate sold under the name Hest 2T-4S® by the company Heterene.

Mention may also be made of silicone waxes and fluoro waxes.

Use may also be made of the waxes obtained by hydrogenation of olive oil esterified with cetyl alcohol, sold under the names Phytowax® ricin 16L64 and 22L73 by the company Sophim. Such waxes are described, for example, in French Patent Application FR A 2792190.

According to certain embodiments, the at least one second wax may be chosen from at least one wax known as a tacky wax, i.e., a wax having a tack of greater than or equal to 0.7 N.s and a hardness of less than or equal to 3.5 MPa.

The use of at least one tacky wax may make it possible to obtain a cosmetic composition which may apply easily to keratin fibers, which may attach well to the keratin fibers, and which may result in the formation of a smooth, uniform, and thickening make-up.

The at least one tacky wax used may, for example, have a tack ranging from 0.7 N.s to 30 N.s, or a tack greater than or equal to 1 N.s, for example a tack ranging from 1 N.s to 20 N.s. The at least one tacky wax used may, for example, have a tack greater than or equal to 2 N.s, such as a tack ranging from 2 N.s to 10 N.s, or ranging from 2 N.s to 5 N.s.

The tack of the wax may be determined by measuring the change in strength (compressive strength or stretching strength) as a function of time, at 20° C., using the texturometer sold under the name TA-TX2i by the company Rheo, equipped with a moving body made of acrylic polymer in the shape of a cone forming a 45° angle.

The measuring protocol is as follows:

The wax is melted at a temperature equal to the melting point of the wax +10° C. The molten wax is poured into a container 25 mm in diameter and 20 mm deep. The wax is recrystallized at ambient temperature (25° C.) for 24 hours, such that the surface of the wax is flat and smooth, and the wax is then stored for at least 1 hour at 20° C. before the tack is measured.

The moving body of the texturometer is moved at a rate of 0.5 mm/s, and then penetrated into the wax to a penetration depth of 2 mm. When the moving body has penetrated into the wax to a depth of 2 mm, the moving body is kept fixed at this point for 1 second (corresponding to the relaxation time) and is then withdrawn at a rate of 0.5 mm/s.

During the relaxation time, the strength (compressive strength) decreases to zero, and then, during the withdrawal of the moving body, the strength (stretching strength) becomes negative and then again increases towards zero. The tack corresponds to the integral of the curve of the strength as a function of time for the portion of the curve corresponding to the negative values for the strength (stretching strength). The tack value is expressed in N.s.

As disclosed above, the tacky wax which can be used may have a hardness of less than or equal to 3.5 MPa, such as a hardness ranging from 0.01 MPa to 3.5 MPa, for example ranging from 0.05 MPa to 3 MPa, or ranging from 0.1 MPa to 2.5 MPa.

The hardness may be measured according to the protocol described above.

As examples of at least one tacky wax, use may be made of a C₂₀-C₄₀ alkyl(hydroxystearyloxy)stearate (the alkyl group comprising from 20 to 40 carbon atoms), alone or in a mixture, such as a C₂₀-C₄₀ alkyl 12-(12′-hydroxystearyloxy)stearate of formula (I): 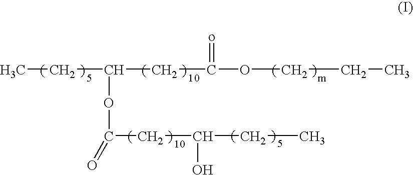
in which m is an integer ranging from 18 to 38, as well as mixtures of compounds of formula (I).

Such a wax is, for example, sold under the names Kester Wax K 82 P® and Kester Wax K 80 P® by the company Koster Keunen.

The compositions disclosed herein may also comprise, in an additional capacity, at least one microwax having a starting melting point of less than or equal to 45° C.

The content of wax having a starting melting point of less than or equal to 45° C. may, in certain embodiments, be present in an amount ranging from 0.1 to 50%, such as from 1 to 45%, or from 5 to 40%, by weight relative to the total weight of the composition.

In the composition disclosed herein, use may be made of a mixture of waxes, for example use may be made of at least one wax referred to as a “conventional-type wax” and at least one wax referred to as a “microwax”.

The at least one wax or the mixture of waxes is present, in the compositions disclosed herein, in the form of a dispersion of particles in the nonaqueous solvent medium.

The overall content of the at least one wax, i.e., comprising both the high starting melting point waxes and the low starting melting point waxes when they are present, may range from 20% to 70%, such as from 22% to 65%, or from 25% to 60%, by weight relative to the total weight of the composition.

The particles of wax may be in varied forms. They may, for example, be substantially spherical.

Volatile Oil

The composition disclosed herein comprises at least one volatile oil.

This at least one volatile oil is capable of forming a continuous phase.

As used herein, the term “oil” is intended to mean a water-insoluble fatty substance that is liquid at ambient temperature and atmospheric pressure.

As used herein, the term “volatile” is intended to mean any compound capable of evaporating on contact with the skin or with other keratin fibers in less than one hour, at ambient temperature and atmospheric pressure. The volatile compound is a volatile cosmetic compound, that is liquid at ambient temperature, having a vapor pressure that is not zero, at ambient temperature and atmospheric pressure, such as having a vapor pressure ranging from 0.13 Pa to 40,000 Pa (10⁻³ to 300 mmHg), or a vapor pressure ranging from 1.3 Pa to 13,000 Pa (0.01 to 100 mmHg), such as a vapor pressure ranging from 1.3 Pa to 1,300 Pa (0.01 to 10 mmHg).

Conversely, as used herein the term “non-volatile” is intended to mean a compound which remains on the skin or other keratin fibers at ambient temperature and atmospheric pressure for at least several hours and which has a vapor pressure of less than 10⁻³ mmHg (0.13 Pa).

As disclosed herein, the at least one volatile oil forms a nonaqueous solvent medium in which it may represent the major constituent. In other words, the at least one volatile oil may represent more than 50% by weight of said nonaqueous solvent medium. For example, the at least one volatile oil may represent at least 60%, for example, at least 70%, and can range up to 100%, by weight relative to the total weight of said nonaqueous solvent medium.

The content of the at least one volatile oil may be between 5% and 80%, such as ranging from 15% to 75%, or it may be less than or equal to 70%, such as ranging from 20% to 60%, by weight relative to the total weight of the composition. The at least one volatile oil disclosed herein is cosmetically acceptable. As used herein, the term “cosmetically acceptable” is intended to mean a compound, the use of which is compatible with an application onto keratin fibers, such as the skin.

The composition disclosed herein can comprise a mixture of volatile oils.

The at least one volatile oil may be chosen from at least one of hydrocarbon-based oils, silicone oils, and fluoro oils.

As used herein, the term “hydrocarbon-based oil” is intended to mean an oil containing mainly hydrogen and carbon atoms and, optionally, at least one atom chosen from oxygen, nitrogen, sulphur, and phosphorus atoms. The volatile hydrocarbon-based oils can be chosen from hydrocarbon-based oils having from 8 to 16 carbon atoms, for example branched C₈-C₁₆ alkanes, such as petroleum-based C₈-C₁₆ isoalkanes (also called isoparaffins), for instance isododecane (also called 2,2,4,4,6-pentamethylheptane), isodecane and isohexadecane, and for example the oils sold under the trade names Isopars® and Permetyls®, branched C₈-C₁₆ esters, isohexyl neopentanoate, and mixtures thereof. Other volatile hydrocarbon-based oils, such as petroleum distillates, for example those sold under the name Shell Solt® by the company Shell, can also be used.

As volatile oils, use may also be made of volatile silicones, such as, for example, linear or cyclic volatile silicone oils, for example those having a viscosity less than or equal to 6 centistokes (6×10⁻⁶ m²/s), and having from 2 to 10 silicon atoms, wherein these silicones optionally comprise a group chosen from alkyl groups having from 1 to 22 carbon atoms and alkoxy groups having from 1 to 22 carbon atoms. As a volatile silicone oil which can be used in accordance with certain embodiments, mention may be made of at least one of octamethylcyclotetrasiloxane, decamethylcyclopentasiloxane, dodecamethylcyclohexasiloxane, heptamethylhexyltrisiloxane, heptamethyloctyltrisiloxane, hexamethyldisiloxane, octamethyltrisiloxane, decamethyltetrasiloxane, and dodecamethylpentasiloxane.

According to certain embodiments disclosed herein, the at least one volatile oil is chosen from at least one hydrocarbon-based volatile oil having from 8 to 16 carbon atoms.

In the compositions disclosed herein, the at least one volatile oil can be in a mixture with another water-insoluble compound that is liquid at ambient temperature and atmospheric pressure.

According to one embodiment, the compositions may comprise at least one volatile organic solvent, for example a fluorinated solvent, such as nonafluoromethoxybutane and perfluoromethylcyclopentane.

The nonaqueous solvent medium of the compositions disclosed herein may also comprise at least one non-volatile, water-insoluble compound that is liquid at ambient temperature, such as at least one non-volatile oil which may be chosen from non-volatile hydrocarbon-based oils, silicone oils, and fluoro oils.

As non-volatile hydrocarbon-based oils that can be used herein, mention may be made of:
hydrocarbon-based oils of plant origin, such as the triglycerides comprising esters of fatty acids and of glycerol, in which the fatty acids may have varied chain lengths ranging from C₄ to C₂₄, wherein these chains may be linear or branched, and saturated or unsaturated; these oils may be, for example, wheatgerm oil, sunflower oil, grapeseed oil, sesame oil, corn oil, apricot oil, castor oil, shea butter, avocado oil, olive oil, soybean oil, sweet almond oil, palm oil, rapeseed oil, cottonseed oil, hazelnut oil, macadamia oil, jojoba oil, alfalfa oil, poppy oil, pumpkin oil, sesame oil, marrow oil, rapeseed oil, blackcurrant oil, evening primrose oil, millet oil, barley oil, quinoa oil, rye oil, safflower oil, candlenut oil, passionflower oil, musk rose oil, and caprylic/capric acid triglycerides such as those sold by the company Stearineries Dubois and those sold under the names Miglyol® 810, Miglyol® 812, and Miglyol® 818 by the company Dynamit Nobel;
synthetic ethers having from 10 to 40 carbon atoms;
linear or branched hydrocarbons of mineral or synthetic origin, such as at least one of petroleum jelly, polydecenes, hydrogenated polyisobutene such as parleam, and squalane;
synthetic esters such as the oils of formula R₁COOR₂ in which R₁ represents the residue of a linear or branched fatty acid having from 1 to 40 carbon atoms and R₂ represents a hydrocarbon-based, optionally branched, chain having from 1 to 40 carbon atoms, with the proviso that the sum of the carbon atoms of R₁+R₂ is greater than or equal to 10, for instance purcellin oil (cetostearyl octanoate), isopropyl myristate, isopropyl palmitate, C₁₂ to C₁₅ alcohol benzoate, hexyl laurate, diisopropyl adipate, isononyl isononanoate, 2-ethylhexyl palmitate, isostearyl isostearate, octanoates, decanoates of alcohols, ricinoleates of alcohols, decanoates of polyalcohols, and ricinoleates of polyalcohols, such as propylene glycol dioctanoate; hydroxylated esters such as isostearyl lactate and diisostearyl maleate, and pentaerythrityl esters;
fatty alcohols that are liquid at ambient temperature and contain a branched and/or unsaturated carbon chain having from 12 to 26 carbon atoms, such as octyl dodecanol, isostearyl alcohol, oleyl alcohol, 2-hexyldecanol, 2-butyloctanol, and 2-undecylpentadecanol;
higher fatty acids such as at least one of oleic acid, linoleic acid, and linolenic acid.

The non-volatile silicone oils which can be used in the compositions disclosed herein can be non-volatile polydimethylsiloxanes (PDMS); polydimethyl-siloxanes comprising alkyl or alkoxy groups which are pendant or at the end of a silicone chain, these groups each having from 2 to 24 carbon atoms; and phenyl silicones, such as phenyl trimethicones, phenyl dimethicones, phenyltrimethylsiloxydiphenylsiloxanes, diphenyl dimethicones, diphenylmethyldiphenyltrisiloxanes, and 2-phenylethyl trimethylsiloxysilicates.

The fluoro oils which can be used in the compositions disclosed herein may be chosen from fluorosilicone oils, fluoro polyethers, and fluorosilicones, as described for example in European Patent Application No. EP A 847752.

The non-volatile, water-insoluble compound that is liquid at ambient temperature may, in certain embodiments, be present in an amount ranging from 0.01 to 30% by weight, such as from 0.1 to 25% by weight, relative to the total weight of the composition.

Water and/or at Least One Water-Soluble Solvent

According to one embodiment disclosed herein, the compositions are free of water and of water-soluble solvents.

According to another embodiment disclosed herein, the compositions, for example the make-up compositions for keratin fibers, comprise a total content of water and/or at least one water-soluble solvent of less than or equal to 20% by weight relative to the weight of the composition.

The term “water-soluble solvent” denotes a compound that is liquid at ambient temperature and water-miscible (i.e., a water-miscibility greater than 500/% by weight at 25° C. and atmospheric pressure).

The at least one water-soluble solvent which can optionally be used in the compositions disclosed herein may also be volatile.

Among the at least one water-soluble solvent which can be used in the compositions disclosed herein, mention may be made of lower monoalcohols having from 1 to 5 carbon atoms, such as ethanol and isopropanol; glycols having from 2 to 8 carbon atoms, such as ethylene glycol, propylene glycol, 1,3-butylene glycol and dipropylene glycol; C₃ and C₄ ketones; and C₂ to C₄ aldehydes.

The water and/or the at least one water-soluble solvent can be introduced as such into the formulation or can be incorporated therein by means of at least one ingredient making up said composition. Thus, for example, water can be introduced into the composition by means of the introduction of latex or of pseudolatex, i.e., of an aqueous dispersion of polymer particles.

The presence of water and/or of at least one water-soluble solvent in said compositions may increase the adhesion of the composition to its support. In fact, the greater the amount of the nonaqueous solvent, for example, the volatile oil, the more slippery the application to the support, due to the mainly “oily” nature of the composition. Partial substitution of the nonaqueous solvent with at least one water-soluble solvent may decrease this effect and thus increase the adhesion to the support. The film obtained after drying may then be thicker.

In these embodiments of the compositions disclosed herein, the content of water and/or of at least one water-soluble solvent may be present in an amount that is greater than or equal to 0.5%, such as an amount ranging from 1 to 18%, or from 2 to 15%, by weight relative to the total weight of the composition.

Polymer(s) Soluble in the at Least One Volatile Oil and Having at Least One Crystallizable Portion

According to some embodiments, the compositions disclosed herein comprise at least one polymer soluble in said at least one volatile oil and having at least one crystallizable portion.

The expression “polymer soluble in said volatile oil” is intended to mean a polymer which, when it is introduced alone in an amount of solids of at least greater than 0.01% by weight and for an amount corresponding to that envisaged for the desired final composition, is soluble in said at least one volatile oil at ambient temperature, generally of the order of 25° C., and under atmospheric pressure (750 mmHg, i.e., 10⁵ Pa).

As used herein, the term “polymer” denotes a compound having at least two repeating units, for example at least three repeating units, at least ten repeating units, or at least fifteen repeating units. The at least one polymer disclosed herein is generally made up of at least two repeating units which are different in nature (copolymer). The at least one polymer as disclosed herein may be of synthetic origin and is characterized by molar masses ranging from 200 to 1,000,000 g/mol, such as from 500 to 500,000 g/mol, or from 1,000 to 300,000 g/mol.

The at least one polymer which can be used herein may be a copolymer that is solubilized and non-crystallized in the at least one volatile oil at ambient temperature, and which comprises at least one crystallizable portion denoted A and at least one non-crystallizable, termed amorphous, portion denoted B.

By virtue of this structure, the at least one polymer may have both an affinity for the at least one wax due to portion A and an affinity for the at least one volatile oil due to portion B, and therefore may contribute, in this capacity, to the dispersion of the at least one wax in the at least one volatile oil.

The crystallizable portion of the at least one polymer which can be used as disclosed herein may be present in an amount of at least 5%, such as at least 10%, and no more than 50%, for example an amount ranging from 30 to 50%, by weight of the total weight of each polymer.

The crystallizable portion A of the at least one copolymer which can be used according to certain embodiments can be represented by a pendant chain linked to the backbone of said at least one polymer and/or a sequence integrated directly into this backbone and/or at least one end chain. This at least one copolymer may have any chemical structure, for example chemical structures chosen from random, block, grafted, and dendrimeric copolymers.

Similarly, the amorphous portion of the at least one copolymer which can be used may be represented by a pendant chain linked to the backbone of said at least one copolymer and/or a sequence integrated directly into this backbone and/or at least one end chain.

As used herein, the term or the expression:
“crystallizable portion A” is intended to denote a series of at least 5 repeating units, such that, if one took the homopolymer corresponding to this repeating unit, it would be characterized by a degree of crystallinity of greater than 30%,
“amorphous portion B” is intended to denote a series of at least 5 repeating units, such that, if one took the homopolymer corresponding to this repeat unit, it would be characterized by a degree of crystallinity of less than 5%, such as zero,
“sequence integrated into the backbone” is intended to denote a group of atoms comprising a repeating monomer unit, which are part of the main chain of the polymer,
“pendant chain or side group” is intended to mean a group of atoms representing a branching at the level of the backbone of the polymer, and
“end chain” is intended to denote a group of atoms located at at least one of the ends of the backbone.

a) Random Copolymers

The random copolymers may be polymers comprising at least one crystallizable pendant chain which comprises units resulting from the polymerization of at least two monomers, at least one of which has a crystallizable hydrophobic side chain referred to as X which can be represented by formula II: 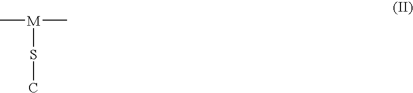
wherein M is an atom of the polymeric backbone, S is a spacer and C is a crystallizable group.

The crystallizable “—S—C” chains may be aliphatic or aromatic, linear, branched or cyclic, and optionally fluorinated or perfluorinated. “S” may, for example, represent a linear, branched or cyclic group chosen from (CH₂)ₙ, (CH₂CH₂O)ₙ, and (CH₂O), wherein n is chosen from an integer ranging from 0 to 22. In certain embodiments, “S” is a linear group. In certain embodiments, “S” and “C” may be different.

When the crystallizable “—S—C” chains are hydrocarbon-based aliphatic chains, they comprise hydrocarbon-based alkyl chains having at least 11 carbon atoms, and no more than 40 carbon atoms, for example no more than 24 carbon atoms. The crystallizable “—S—C” chains may be chosen from aliphatic chains having at least 12 carbon atoms and alkyl chains having at least 12 carbon atoms. For example, they may be C₁₂-C₂₄ alkyl chains. When they are fluorinated or perfluorinated alkyl chains, they comprise at least 6 fluorinated carbon atoms, and for example at least 11 carbon atoms, at least 6 carbon atoms of which are fluorinated.

As an example of polymers comprising (a) at least one crystallizable pendant chain, mention may be made of those comprising units resulting from the polymerization of at least one of the following monomers: saturated alkyl(meth)acrylates with a C₁₂-C₂₄ alkyl group, perfluoroalkyl(meth)acrylates with a C₁₂-C₁₅ perfluoroalkyl group, N-alkyl(meth)acrylamides with a C₁₂ to C₂₄ alkyl group, optionally with a fluorine atom, vinyl esters containing alkyl or perfluoro(alkyl) chains with a C₁₂ to C₂₄ alkyl group (with at least 6 fluorine atoms per perfluoroalkyl chain), allyl esters containing alkyl or perfluoro(alkyl) chains with a C₁₂ to C₂₄ alkyl group (with at least 6 fluorine atoms per perfluoroalkyl chain), vinyl ethers containing alkyl or perfluoro(alkyl) chains with the C₁₂ to C₂₄ alkyl group and at least 6 fluorine atoms per perfluoroalkyl chain, C₁₂ to C₂₄ alpha-olefins such as, for example, octadecene, and para-alkyl styrenes with an alkyl group comprising from 12 to 24 carbon atoms.

By way of illustration of the at least one polymer which can be used as disclosed herein, mention may be made of the copolymers of linear and saturated C₁₂ to C₃₀ alkyl(meth)acrylates, constituting the crystallizable portion A and linear C₄ to C₁₀ or branched or cyclic and/or unsaturated C₄ to C₃₀ alkyl(meth)acrylates constituting the amorphous portion B.

Among the copolymers of vinyl esters containing linear and saturated C₁₂ to C₃₀ alkyl groups constituting the crystallizable portion A and of vinyl esters containing linear C₄ to C₁₀ or branched or cyclic and/or unsaturated C₄ to C₃₀ alkyl groups constituting the amorphous portion B, mention may be made of copolymers chosen from copolymers of vinyl acetate, copolymers of vinyl stearate, and copolymers of allyl stearate, such as the copolymer of allyl stearate and of vinyl acetate sold under the name Mexomere PQ® by the company Chimex.

When the at least one polymer results from a polycondensation, the hydrocarbon-based and/or fluorinated crystallizable chains as defined above are carried by a monomer chosen from diacids, diols, diamines, and diisocyanates.

b) Block Copolymers

These polymers comprise at least two types of sequences which are chemically different in nature, one of which is crystallizable and constitutes portion A. In the case of the block copolymers, at least one of the amorphous sequences B should be soluble in the at least one volatile oil.

Mention may be made, for example, of:
block copolymers of olefin and block copolymers of cycloolefin containing a crystallizable chain, such as those derived from the block polymerization:
   of at least one entity chosen from cyclobutene, cyclohexene, cyclooctene, norbornene (i.e., bicyclo[2,2,1]-2-heptene), 5-methylnorbornene, 5-ethylnorbornene, 5,6-dimethylnorbornene, 5,5,6-trimethyl norbornene, 5-ethylidenenorbornene, 5-phenyl norbonene, 5-benzylnorbornene, 5-vinylnorbornene, 1,4,5,8-dimethano-1,2,3,4,4a,5,8a-octahydronaphthalene, and dicyclopentadiene,
   with at least one entity chosen from ethylene, propylene, 1-butene, 3-methyl-1-butene, 1-hexene, 4-methyl-1-pentene, 1-octene, 1-decene, and 1-eicosene;
the block copolymers block or multiblock hydrogenated poly-(butyleneterephthalate)-b-poly(isoprene), mentioned in the article “Study of morphological and mechanical properties of PP/PBT” by B. Boutevin et al., Polymer Bulletin, 34,117-123 (1995),
the block copolymers poly(ethylene)-b-copoly(ethylene/propylene) mentioned in the articles “Morphology of semi-crystalline block copolymers of ethylene-(ethylene-alt-propylene)” by P. Rangarajan et al., Macromolecules, 26, 4640-4645 (1993) and “Polymer aggregates with crystalline cores: the system poly(ethylene)-poly(ethylene-propylene)” by P. Richter et al., Macromolecules, 30, 1053-1068 (1997); and
the block copolymers poly(ethylene)-b-poly(ethylethylene) mentioned in the general article “Cristallization in block copolymers” by I. W. Hamley, Advances in Polymer Science, vol 148, 113-137 (1999).

These polymers may have a single crystallizable sequence or a repeat of crystallizable sequences. In the latter case, these crystallizable sequences can be chemically identical or different in nature.

c) Copolymers Containing Crystallizable End Sequences

In this category, mention may be made, for example, of:
polyamide polycondensates resulting from condensation between (a) at least one acid chosen from dicarboxylic acids comprising at least 32 carbon atoms, such as dimeric fatty acids, and (b) an alkylenediamine, such as ethylenediamine, in which the polyamide polymer comprises at least one end carboxylic acid group which is esterified or amidated with at least one monoalcohol or monoamine comprising from 12 to 30 linear and saturated carbon atoms, for example the copolymers of ethylenediamine/stearyl dilinoleate such as those sold under the name Uniclear 100 VG® by the company Arizona Chemical; and
lipophilic polyester polycondensates in which the ends are esterified with a crystallizable acid or alcohol comprising a linear, saturated C₁₂ to C₃₀ carbon chain, for example 12-polyhydroxystearic acid, in which at least one of the ends is esterified with stearic acid, such as Solsperse 21000® sold by the company Avecia.

By way of illustration of the copolymers which can be used according to the present disclosure, mention may be made of ethylene/vinyl acetate copolymers, ethylene/maleic anhydride copolymers, hydrogenated butadiene/isoprene block copolymers, and ethylene/maleic anhydride/vinyl acetate terpolymers.

The at least one polymer soluble in the at least one volatile oil and having at least one crystallizable portion can be present in the composition disclosed herein in an amount ranging from 0.01% to 30%, such as from 0.1 to 20%, or from 1 to 10%, by weight relative to the total weight of the composition.

Film-Forming Polymer

According to one embodiment, the composition disclosed herein can comprise at least one film-forming polymer.

As used herein, the term “film-forming polymer” is intended to mean a polymer capable of forming, by itself or in the presence of an auxiliary film-forming agent, a continuous film that adheres to a support, such as to keratin materials.

Classified in this category are polymers that are generally liposoluble, comprising less than 30% by weight of a portion that is crystallizable under the conditions disclosed herein, for example polymers not containing any portion that is crystallisable under the conditions disclosed herein.

Among the film-forming polymers which can be used, mention may be made of synthetic free-radical polymers, synthetic polycondensate polymers, polymers of natural origin, and mixtures thereof.

By way of example of a liposoluble polymer, mention may be made of copolymers of vinyl ester (the vinyl group being directly-linked to the oxygen atom of the ester group and the vinyl ester having a saturated, linear or branched hydrocarbon-based radical having from 1 to 24 carbon atoms, linked to the carbonyl of the ester group) and of at least one other monomer, which may be a vinyl ester (different from the vinyl ester already present), alkyl vinyl ethers (in which the alkyl group comprises from 2 to 24 carbon atoms), allyl esters (having a saturated, linear or branched hydrocarbon-based radical of from 1 to 24 carbon atoms, linked to the carbonyl of the ester group), and methallyl esters (having a saturated, linear or branched hydrocarbon-based radical of from 1 to 24 carbon atoms, linked to the carbonyl of the ester group). The at least one copolymer can be crosslinked by means of crosslinking agents, which can chosen from vinyl crosslinking agents, allyl crosslinking agents, and methallyl crosslinking agents, such as tetraallyloxyethane, divinylbenzene, divinyl octanedioate, divinyl dodecanedioate, and divinyl octadecanedioate.

As examples of the at least one copolymer which may be used herein, mention may be made of the following copolymers: vinyl acetate/vinyl laurate, vinyl propionate/allyl laurate, vinyl propionate/vinyl laurate, and allyl 2,2-dimethyl pentanoate/vinyl laurate.

As liposoluble film-forming polymers, mention may also be made of liposoluble homopolymers, such as those resulting from the homopolymerization of at least one entity chosen from vinyl esters having 9 to 22 carbon atoms, alkyl acrylates, and methacrylates, the alkyl radicals having 2 to 24 carbon atoms.

As examples of liposoluble homopolymers, mention may be made of: poly(vinyl laurates) and poly(lauryl(meth)acrylates), wherein these poly(meth)acrylates are optionally crosslinked by means of ethylene glycol dimethacrylate or tetraethylene glycol dimethacrylate.

The liposoluble copolymers and homopolymers defined above are known and described, for example, in French Patent Application FR A 2232303. They may have a weight-average molecular weight ranging from 2,000 to 500,000, for example from 4,000 to 200,000.

As liposoluble film-forming polymers which can be used, mention may also be made of polyalkylenes, for example copolymers of C₂ to C₂₀ alkenes, such as polybutene, alkylcelluloses with a linear or branched C₁ to C₈ alkyl radical which are optionally saturated, such as ethylcellulose and propylcellulose, copolymers of vinylpyrrolidone (VP), and copolymers of C₂ to C₄₀ alkense, for example C₃ to C₂₀ alkenes. By way of example of VP copolymers which can be used, mention may be made of copolymers of VP/vinyl acetate, VP/ethyl methacrylate, butyl polyvinylpyrrolidone (PVP), VP/ethyl methacrylate/methacrylic acid, VP/eicosene, VP/hexadecene, VP/triacontene, VP/styrene, and VP/acrylic acid/lauryl methacrylate.

The at least one film-forming polymer may also be present in the composition in the form of particles in a dispersion in an aqueous phase or in a nonaqueous solvent phase, generally known as latex or pseudolatex. The techniques for preparing these dispersions are well known to those skilled in the art.

As an aqueous dispersion of at least one film-forming polymer, use may be made of the acrylic dispersions sold under the names Neocryl XK-90®, Neocryl A-1070®, Neocryl A-1090®, Neocryl BT-62®, Neocryl A 1079®, and Neocryl A-523® by the company Avecia-Neoresins; Dow Latex 432® by the company Dow Chemical; Daitosol 5000 AD® by the company Daito Kasey Kogyo; the aqueous dispersions of polyurethane sold under the names Neorez R-981® and Neorez R-974° by the company Avecia-Neoresins; Avalure UR-405®, Avalure UR-410®, Avalure UR-425®, Avalure UR-450®, Sancure 875®, Sancure 861®, Sancure 878® and Sancure 2060® by the company Goodrich; Impranil 85® by the company Bayer; Aquamere H-1511® by the company Hydromer; the sulphopolyesters sold under the trade name Eastman AQ® by the company Eastman Chemical Products; the vinyl dispersions such as Mexomère PAM and also the acrylic dispersions in isodecane such as Mexomere PAP sold by the company Chimex.

The at least one film-forming polymer may be present in the composition in an amount of solids ranging from 0.1% to 30% by weight relative to the total weight of the composition, such as from 0.5% to 25% by weight, or from 1% to 20% by weight.

The composition disclosed herein may further comprise at least one plasticizer which may promote the formation of a film with the film-forming polymer. Such a plasticizer can be chosen from all the compounds known to those skilled in the art that may be capable of performing the desired function.

Dyestuff

The composition disclosed herein may also comprise at least one dyestuff, such as pulverulent materials, liposoluble dyes, and water-soluble dyes.

The pulverulent dyestuffs can be chosen from pigments and pearlescent agents.

The pigments can be chosen from white or colored, mineral and/or organic, coated or uncoated pigments. Among the mineral pigments, mention may be made of titanium dioxide, optionally surface-treated, zirconium oxide, zinc oxide, cerium oxide, iron oxide and chromium oxide, manganese violet, ultramarine blue, chromium hydrate, and ferric blue. Among the organic pigments, mention may be made of carbon black, D & C pigments, cochenille carmine lakes, barium lakes, strontium lakes, calcium lakes, and aluminium lakes.

The pearlescent agents can be chosen from white pearlescent pigments such as mica coated with titanium and mica coated with bismuth oxychloride, colored pearlescent pigments such as titanium mica with iron oxides, titanium mica with ferric blue, titanium mica with chromium oxide, titanium mica with an organic pigment of the abovementioned type, and also pearlescent pigments based on bismuth oxychloride.

The liposoluble dyes are, for example, Sudan red, D&C Red 17, D&C Green 6, β-carotene, soybean oil, Sudan brown, D&C Yellow 11, D&C Violet 2, D&C Orange 5, quinoline yellow, and annatto.

These dyestuffs can be present in an amount ranging from 0.01 to 30% by weight relative to the total weight of the composition.

Fillers

The composition disclosed herein may also comprise at least one filler.

The fillers can be chosen from those that are well known to those skilled in the art and commonly used in cosmetic compositions. The fillers can be mineral or organic, lamellar or spherical. Mention may be made of talc, mica, silica, kaolin, polyamide powders such as the Nylon® sold under the name Orgasol® by the company Atochem, poly-β-alanine powders, polyethylene powders, powders of tetrafluoroethylene polymers such as Teflon®, lauroyllysine, starch, boron nitride, expanded hollow polymer microspheres, such as those of polyvinylidene chloride/acrylonitrile, for instance those sold under the name Expancel® by the company Nobel Industrie, acrylic powders such as those sold under the name Polytrap® by the company Dow Corning, poly(methyl methacrylate) particles and silicone resin microbeads (Tospearls® from Toshiba, for example), precipitated calcium carbonate, magnesium carbonate and magnesium hydrocarbonate, hydroxyapatite, hollow silica microspheres (Silica Beads® from Maprecos), glass or ceramic microcapsules, metal soaps derived from organic carboxylic acids having from 8 to 22 carbon atoms, for example having from 12 to 18 carbon atoms, for example zinc stearate, magnesium stearate, lithium stearate, zinc laurate, and magnesium myristate.

The fillers may be present in an amount ranging from 0.1 to 25%, such as from 1 to 20%, by weight relative to the total weight of the composition.

The composition disclosed herein may also comprise any cosmetically acceptable additive, for example additives chosen from those usually used in cosmetics, such as antioxidants, preserving agents, fragrances, neutralizing agents, plasticizers, thickening or gelling agents, fibers, cosmetic active agents, and mixtures thereof.

The gelling agents which can be used in the compositions according to the invention may be lipophilic and may be organic or mineral, polymeric or molecular.

As mineral lipophilic gelling agent, mention may be made of optionally modified clays, such as hectorites modified with a C₁₀ to C₂₂ fatty acid ammonium chloride, for instance hectorite modified with distearyldimethylammonium chloride such as, for example, that sold under the name Bentone 38V® by the company Elementis.

Mention may also be made of surface-hydrophobic, optionally treated pyrogenic silica, the particle size of which is less than 1 μm. It may be possible to chemically modify the surface of the silica, by chemical reaction generating a decrease in the number of silanol groups present at the surface of the silica. Silanol groups can be substituted with hydrophobic groups: a hydrophobic silica is then obtained. The hydrophobic groups can be:
trimethylsiloxyl groups, which may be obtained by treatment of pyrogenic silica in the presence of hexamethyldisilazane. Silicas thus treated are called “Silica silylate” according to the CTFA (6^{th} edition, 1995). They are, for example, sold under the names Aerosil R812® by the company Degussa and CAB-O-SIL TS-530® by the company Cabot,
dimethylsilyloxyl groups and polydimethylsiloxane groups, which may be obtained by treatment of pyrogenic silica in the presence of at least one of polydimethylsiloxane and dimethyldichlorosilane. Silicas thus treated are called “silica dimethyl silylate” according to the CTFA (6^{th} edition, 1995). They are, for example, sold under the names Aerosil R972® and Aerosil R974® by the company Degussa and CAB-O-SIL TS-610® and CAB-O-SIL TS-720® by the company Cabot.

The hydrophobic pyrogenic silica may have a particle size which can be nanometric to micrometric, for example ranging from 5 to 200 nm.

The polymeric organic lipophilic gelling agents are, for example, partially or totally crosslinked elastomeric organopolysiloxanes, having a three-dimensional structure, such as those sold under the names KSG6, KSG16 and KSG18 by the company Shin-Etsu, Trefil E-505C® and Trefil E-506C® by the company Dow-Corning, Gransil® SR-CYC, SR DMF10, SR-DC556, SR 5CYC gel, SR DMF 10 gel and SR DC 556 gel by the company Grant Industries, SF 1204 and JK 113 by the company General Electric; ethylcellulose, for instance that sold under the name Ethocel® by the company Dow Chemical; galactomannans comprising from one to six, for example from two to four, hydroxyl groups per monosaccharide, substituted with a saturated or unsaturated alkyl chain, for instance guar gum alkylated with C₁ to C₆ alkyl chains, such as C₁ to C₃, alkyl chains, and mixtures thereof; the “diblock”- or “triblock”-type block copolymers of the type polystyrene/polyisoprene and polystyrene/polybutadiene, such as those sold under the name Luvitol HSB® by the company BASF, of the type polystyrene/copoly(ethylene-propylene) such as those sold under the name Kratone by the company Shell Chemical Co, and of the type polystyrene/copoly(ethylene-butylene).

Among the gelling agents which can be used in the compositions disclosed herein, mention may also be made of the esters of dextrin and of a fatty acid, such as dextrin palmitates, such as those sold under the names Rheopearl TL® or Rheopearl KL® by the company Chiba Flour.

The compositions disclosed herein can also comprise fibers which make it possible to improve the lengthening effect.

The term “fiber” should be understood to mean an object of length L and of diameter D such as L is much greater than D, D being the diameter of the circle within which the cross-section of the fiber falls. In certain embodiments, the ratio L/D (or shape factor) ranges from 3.5 to 2500, for example from 5 to 500, or from 5 to 150.

The fibers which can be used in the compositions disclosed herein can be chosen from synthetic or natural fibers, of mineral or organic origin. The fibers can be short or long, and single or organized, for example braided, hollow or solid. They may have any shape, for example they may have a circular or polygonal (square, hexagonal or octagonal) cross-section, according to the specific application envisaged. In certain embodiments, their ends are blunt or polished to prevent injury.

In certain embodiments, the fibers have a length ranging from 1 μm to 10 mm, such as from 0.1 mm to 5 mm, or from 1 mm to 3.5 mm. Their cross-section can be included in a circle having a diameter ranging from 2 nm to 500 μm, for example a circle having a diameter ranging from 100 nm to 100 μm, or from 1 μm to 50 μm. The weight or yarn count of the fibers is often given in denier or decitex, and represents the weight in grams per 9 km of yarn. The fibers disclosed herein may, for example, have a yarn count ranging from 0.15 to 30 denier, such as from 0.18 to 18 denier.

The fibers can be those used in the manufacture of textiles, for example silk fiber, cotton fiber, wool fiber, flax fiber, cellulose fiber (for example fiber from wood, vegetables, and algae), rayon fiber, polyamide (Nylon®) fiber, viscose fiber, acetate fiber such as rayon acetate fiber, poly(p-phenyleneterephthalamide) (or aramide) fiber such as Kevlar® fiber, acrylic polymer fiber such as poly(methyl methacrylate) fiber and poly(2-hydroxyethyl methacrylate) fiber, polyolefin fiber such as polyethylene and polypropylene fiber, glass fiber, silica fiber, carbon fiber such as fiber in graphite form, polytetrafluoroethylene (such as Teflon®) fiber, insoluble collagen fiber, polyester fiber, polyvinyl chloride fiber, polyvinylidene chloride fiber, polyvinyl alcohol fiber, polyacrylonitrile fiber, chitosan fiber, polyurethane fiber, polyethylene phthalate fiber, and fibers made up of a mixture of polymers such as those mentioned above, for instance polyamide/polyester fibers.

The fibers used in surgery may also be used, for instance the resorbable synthetic fibers prepared from glycolic acid and caprolactone (Monocryl® from Johnson & Johnson); resorbable synthetic fibers of the type which is a copolymer of lactic acid and of glycolic acid (Vicryl® from Johnson & Johnson); polyterephthalic ester fibers (Ethibond® from Johnson & Johnson) and stainless steel threads (Acier® from Johnson & Johnson).

Moreover, the fibers may optionally be surface-treated and coated. As coated fibers which can be used, mention may be made of polyamide fibers coated with copper sulphide to give an anti-static effect (for example R-STAT from Rhodia) or another polymer enabling a particular organization of the fibers (specific surface treatment) or surface treatment inducing color/hologram effects (Lurex® fiber from Sildorex, for example).

Fibers of synthetic origin, for example organic fibers, such as those used in surgery, may be used. Water-insoluble fibers may also be used.

The fibers which can be used in the compositions disclosed herein may, for example, be chosen from at least one of polyamide fibers, cellulose fibers, poly(p-phenyleneterephtalamide) fibers, and polyethylene fibers. The length (L) of the fibers can range from 0.1 mm to 5 mm, for example from 0.25 mm to 1.6 mm, and their mean diameter can range from 1 μm to 50 μm. According to certain embodiments, the polyamide fibers sold by Etablissements P. Bonte under the name Polyamide 0.9 Dtex 3 mm, having a mean diameter of 6 μm, a yarn count of 0.9 dtex and a length ranging from 0.3 mm to 5 mm, can be used. Cellulose (or rayon) fibers with a mean diameter of 50 μm and a length ranging from 0.5 mm to 6 mm may also be used, for instance those sold under the name Natural rayon flock fiber RC1BE-N003-M04 by the company Claremont Flock. Polyethylene fibers, for instance those sold under the name Shurt Stuff 13 099 F® by the company Mini Fibers, can also be used.

The compositions disclosed herein may also comprise “rigid” fibers, as opposed to the fibers mentioned above, which are not rigid fibers.

The rigid fibers, which are initially substantially straight, when placed in a dispersing medium, do not undergo a substantial change in shape, which is reflected by the angular condition defined below, reflecting a shape which can be described as still substantially straight and linear. This angle condition reflects the stiffness of the fibers, which is difficult to express by another parameter for objects that are as small as the rigid fibers.

The stiffness of the fibers is reflected by the following angular condition: at least 50%, for example at least 75% or at least 90%, in numerical terms, of the fibers are such that the angle formed between the tangent to the longitudinal central axis of the fiber and the straight line connecting said end to the point on the longitudinal central axis of the fiber corresponding to half the length of the fiber is less than 15°, and the angle formed between the tangent to the longitudinal central axis of the fiber at a point halfway along the fiber and the straight line connecting one of the ends to the point on the longitudinal central axis of the fiber corresponding to half the length of the fiber is less than or equal to 15° for the same fiber length ranging from 0.8 mm to 5 mm, for example ranging from 1 mm to 4 mm, from 1 mm to 3 mm, or having a length of 2 mm.

The angle mentioned above may be measured at the two ends of the fiber and at a point halfway along the fiber; in other words, three measurements are taken in this case, and the average of the angles measured is less than or equal to 15°.

The tangent, at any point on the fiber, may form an angle of less than 15°.

According to the present disclosure, the angle formed by the tangent at a point on the fiber is the angle formed between the tangent to the longitudinal central axis of the fiber at said point on the fiber and the straight line connecting the end of the fiber that is closest to said point to the point on the longitudinal central axis of the fiber corresponding to half the length of the fiber.

The rigid fibers which can be used in the compositions disclosed herein may have, for example, the same or substantially the same fiber length.

For example, when a medium in which the rigid fibers are dispersed at a fiber concentration of 1% by weight is observed under a microscope, with an objective lens allowing a magnification of 2.5 and with a full-field vision, a numerical majority of rigid fibers, i.e, at least 50%, for example at least 75% or at least 90%, in numerical terms, of the rigid fibers must satisfy the angular condition defined above. The measurement resulting in the value for the angle is performed for the same length of fibers, this length ranging from 0.8 mm to 5 mm, for example ranging from 1 to 4 mm, from 1 to 3 mm, or a length of 2 mm.

The medium in which the observation is performed is a dispersing medium that ensures adequate dispersion of the rigid fibers, for example water or an aqueous gel of clay or of associative polyurethane. A direct observation of the composition containing the rigid fibers can be performed. A sample of the composition or of the dispersion prepared is placed between a slide and cover slip for observation under a microscope with an objective lens allowing a magnification of 2.5 and with a full-field vision. Full-field vision may make it possible to view the fibers in their entirety.

The rigid fibers can be chosen from fibers of a synthetic polymer chosen from polyesters, polyurethanes, acrylic polymers, polyolefins, polyamides such as non-aromatic polyamides, and aromatic polyimideamides.

As examples of rigid fibers, mention may be made of:
polyester fibers, such as those obtained by chopping yarns sold under the names Fibre 255-100-R11-242T Taille 3 mm (eight-lobed cross section), Fibre 265-34-R11-56T Taille 3 mm (round cross section), and Fibre Coolmax 50-34-591 Taille 3 mm (four-lobed cross section) by the company Dupont de Nemours;
polyamide fibers, such as those sold under the names Trilobal Nylon 0.120-1.8 DPF®; Trilobal Nylon 0.120-18 DPF®; and Nylon 0.120-6 DPF® by the company Cellusuede Products; and those obtained by chopping yarns sold under the name Fibre Nomex® Brand 430 Taille 3 mm® by the company Dupont de Nemours;
polyimideamide fibers, such as those sold under the names Kermel® and Kermel Tech® by the company Rhodia;
poly(p-phenyleneterephtalamide) (or aramide) fibers sold for example under the name Kevlar® by the company Dupont de Nemours;
fibers with a multilayer structure comprising alternating layers of polymers chosen from polyesters, acrylic polymers and polyamides, such as those described in European Patent Applications EP A 6921217 and EP A 686858 and U.S. Pat. No. 5,472,798. Such fibers are sold under the names Morphotex® and Teijin Tetron Morphotex® by the company Teijin.

Rigid fibers that may be used, for example, are aromatic polyimideamide fibers.

Polyimideamide yarns or fibers which can be used in the compositions disclosed herein are described, for example, in the document by R. Pigeon and P. Allard, Chimie Macromoléculaire Appliquée [Applied macromolecular chemistry], 40/41 (1974), pages 139-158 (No. 600), and in U.S. Pat. No. 3,802,841, French Patent No. FR A 2 079 785, and European Patent Nos. EP A1 0 360 728 and EP A 0 549 494.

Aromatic polyimideamide fibers which may be mentioned are, for example, polyimideamide fibers comprising repeating units of formula: 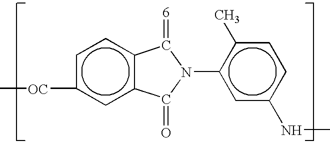
obtained by polycondensation of tolylene diisocyanate and trimellitic anhydride.

The fibers can be present in the compositions in an amount ranging from 0.01% to 10% by weight, relative to the total weight of the composition, for example from 0.1% to 5%, or from 0.3% to 3% by weight, relative to the total weight of the composition.

As cosmetic active agents which can be used in the compositions disclosed herein, mention may be made of emollients, moisturizers, vitamins, and screening agents, for example sunscreens.

Of course, those skilled in the art will take care to choose the optional complementary additives and/or their amounts such that the advantageous properties of the compositions disclosed herein are not, or not substantially, altered by the addition envisaged.

The choice of the optional additives will of course depend on the type of composition envisaged.

The compositions disclosed herein for caring for and/or for making-up the skin and/or the lips may, for example, be provided in a form chosen from a foundation, a blusher, an eye shadow, a lipstick, a care base for the lips, and a care cream for the body and/or the face.

The compositions for making up keratin fibers can be provided in a form chosen from mascaras, products for the eyebrows, eyeliners, and make-up products for the hair. In certain embodiments, the composition is a mascara. The composition may for example be chosen from make-up compositions, compositions to be applied over or under a make up, also called, respectively, “top-coats” or “base-coats”, and compositions for treating the eye lashes.

Characterization of the Solids Content

As used herein, the term “solids content” denotes the content of non-volatile matter.

This solids content, commonly referred to as the “dry extract”, or in its abbreviated form DE, of the compositions disclosed herein may be measured by heating the sample with infrared rays with a wavelength ranging from 2 μm to 3.5 μm. The substances contained in said compositions which have a high vapor pressure evaporate under the effect of this radiation. Measurement of the weight loss of the sample may make it possible to determine the “dry extract” of the composition. These measurements may be made using an LP16 commercial infrared desiccator from Mettler. This technique is fully described, for example, in the machine documentation supplied by Mettler.

The measuring protocol is as follows:

Approximately 1 g of the composition is spread onto a metal crucible. After introducing this crucible into the desiccator, it is subjected to a set temperature of 120° C. for one hour. The wet mass of the sample, corresponding to the initial mass, and the dry mass of the sample, corresponding to the mass after exposure to the radiation, are measured using a precision balance.

The solids content is calculated in the following manner:
Dry extract=100×(dry mass/wet mass).

The values measured using the protocol described above can differ from the corresponding theoretical values by plus or minus 1%.

The compositions disclosed herein may have, for example, a solids content of greater than or equal to 40%, for example greater than or equal to 43%, greater than or equal to 45%, greater than or equal to 47%, or greater than or equal to 49%, by weight relative to the total weight of the composition.

Rheological Characteristics

The compositions disclosed herein can also be characterized by its viscoelastic behavior, for example using various rheological parameters.

In general, a material is said to be viscoelastic when, under the effect of shear, it has both the characteristics of a purely elastic material, i.e., capable of storing energy, and the characteristics of a purely viscous material, i.e., capable of dissipating energy.

In certain embodiments, the viscoelastic behavior of the compositions can be characterized by its modulus of rigidity G, its elasticity δ, and its flow threshold τ_{c}. These parameters are for example defined in the publication “Initiation à la rhéologie” [Introduction to rheology], G. Couarraze and J. L. Grossiord, 2nd edition, 1991, published by Lavoisier-Tec 1 Doc.

These parameters are determined by means of measurements taken at 25° C.±0.5° C. using a Haake RheoStress 600® controlled-stress rheometer from the company Thermorhéo, equipped with a stainless-steel rotor with a plate/plate geometry, the plate having a diameter of 20 mm and a gap (distance between the lower plate, known as the stator plate, on which the composition is deposited, and the upper plate, known as the rotor plate) of 0.3 mm. The two plates are striated to limit phenomena of sliding to the walls of the plates.

The dynamic measurements are made by applying a harmonic variation of the stress. In these experiments, the magnitudes of the shear, of the shear rate, and of the stress are low so as to remain within the limits of the linear viscoelastic range of the material (conditions for evaluating the rheological characteristics of the composition at rest).

The linear viscoelastic range may be defined by the fact that the response of the material (i.e., the strain) is at any moment directly proportional to the value of the applied force (i.e., the stress). In this range, the applied stresses are small and the material undergoes strains without modifying its microscopic structure. Under these conditions, the material is studied “at rest” and non-destructively.

The composition is subjected to a harmonic shear according to a stress τ(t) varying sinusoidally according to a pulse ω (ω=2Πv, v being the frequency of the applied shear). The composition thus sheared undergoes a stress τ(t) and responds according to a strain γ(t) corresponding to microstrains for which the modulus of rigidity varies little as a function of the imposed stress.

The stress τ(t) and the strain γ(t) are defined, respectively, by the following relationships:
τ(t)=τ₀ cos(ω·t) γ(t)=γ₀ cos(ω·t−δ).
τ₀ being the maximum amplitude of the stress and γ₀ being the maximum amplitude of the strain. δ is the dephasing angle between the stress and the strain.

The measurements are taken at a frequency of 1 Hz (v=1 Hz).

The change in the modulus of rigidity G (corresponding to the ratio of τ₀ to γ₀) and in the elasticity δ (corresponding to the dephasing angle of the applied stress relative to the measured strain) as a function of the applied stress τ(t) are thus measured.

The strain of the composition is measured for the stress region in which the variation of the modulus of rigidity G and of the elasticity δ is less than 7% (microstrain region) and the “plateau” parameters Gp and δₚ are thus determined. The threshold stress τ_{c} (corresponding to the minimum force that is necessary to apply to the composition to cause it to flow) is determined from the curve δ=f(τ) and corresponds to the value of τ for which δ(τ_{c})=1.05 δₚ.

The viscoelastic behavior of the compositions disclosed herein is characterized by a plateau modulus of rigidity Gp which may be less than or equal to 50,000 Pa, for example less than or equal to 35,000 Pa, less than or equal to 30,000 Pa, less than or equal to 28,000 Pa, less than or equal to 27,000 Pa, and less than or equal to 25,000 Pa.

The compositions in accordance with embodiments disclosed herein may, moreover, have a flow threshold τ_{c} ranging from 10 Pa to 200 Pa, for example ranging from 20 Pa to 100 Pa.

Preparation Process

The process for preparing the compositions disclosed herein may depend on the nature of the at least one wax used, for example it may depend on whether the at least one wax is a conventional-type wax or a microwax-type wax.

In one embodiment, the at least one wax used is of conventional type as defined above.

In this embodiment, the make-up compositions for keratin fibers may be obtained by heating the at least one wax or a mixture of several waxes to a temperature greater than the melting temperature of the wax having the highest melting point, until complete melting has been obtained, and then blending and cooling continuously to ambient temperature.

The at least one volatile oil can be added during the blending or prior to the implementation thereof.

It appears that blending the composition instead of stirring it according to a conventional process may promote crystallization of the at least one wax in the form of fine crystals forming small particles. It also appears that this blending breaks up the aggregates of particles possibly formed, which may result, in the finished composition, in a substantially uniform dispersion of small wax particles.

The blending operation can be carried out, for example, in a cylinder mill comprising two cylinders turning in opposite directions, between which the paste passes, or for example in a continuous twin-screw blender which may make it possible to obtain a paste of a constant quality in a reproducible manner.

The conditions under which the blending operation can be carried out are described, for example, in French Patent Application FR 94 00756, the content of which is incorporated by reference herein.

When the composition comprises water and/or a water-soluble solvent and/or optional additional ingredients, they can be introduced into the starting products, optionally in the course of the blending during cooling, or into the finished composition.

This mode of preparing the compositions may allow the incorporation of thermosensitive compounds such as certain active agents, given that it allows them to be introduced at a temperature compatible with their stability and by virtue of the small amount of time spent in the blender.

In another embodiment, the at least one wax used is a microwax as defined above.

By virtue of its formulation in the form of particles, such a wax can be used directly at a temperature below its melting temperature. In other words, in this embodiment of preparation of the compositions, the microwax particles are dispersed directly in the continuous phase, and not by forming them therein by means of melting/recrystallization steps.

This step of dispersion of the wax may be carried out at a temperature below the melting temperature of the wax, for example at ambient temperature, which may aid implementation of the preparation process.

The at least one volatile oil is chosen from those defined above. In this embodiment of preparation process, the water and/or the at least one water-soluble solvent and/or the additional ingredients as defined above can be added either to the starting products or to the finished composition.

In yet another embodiment, the process for preparing the compositions uses both at least one conventional-type wax and/or at least one microwax, as defined above. In this embodiment, the at least one conventional-type wax in molten form may be introduced first into the at least one volatile oil, and then the mixture thus obtained is stirred or blended while it cools. The at least one microwax is only introduced when the temperature of the mixture containing the at least one conventional-type wax is below the melting temperature of said at least one microwax or below the melting temperature of the at least one microwax in a mixture of microwaxes which has the lowest melting temperature, for example at ambient temperature.

Here again, the water and/or the at least one water-soluble solvent and the optional additional ingredients can be added, according to the case, either to the starting products or to the finished composition, or when the composition is blended, during cooling.

One embodiment disclosed herein is a cosmetic process for caring for and/or for making-up the skin, in which a composition as defined above is applied to the skin.

The compositions for the skin can be applied, for example, using a fine brush or a brush.

Another embodiment disclosed herein is a process for making-up keratin fibers, in which a composition as defined above is applied to said keratin fibers, such as the eyelashes.

Such compositions can be applied to the eyelashes, using a brush or a comb. In the case of making-up the eyelashes, said composition may be applied with a make-up brush as described in French Patent Nos. FR 2 701 198 and FR 2 605 505 and European Patent Nos. EP 792 603 and EP 663 161, which reinforces the thickening effect.

The examples which follow are presented by way of non-limiting illustration. Other than in the examples, or where otherwise indicated, all numbers expressing quantities of ingredients, reaction conditions, and so forth used in the specification and claims are understood as being modified in all instances by the term “about.” Accordingly, unless indicated to the contrary, the numerical parameters set forth in the following specification and attached claims are approximations that may vary depending upon the desired properties sought to be obtained herein. At the very least, and not as an attempt to limit the application of the doctrine of equivalents to the scope of the claims, each numerical parameter should be construed in light of the number of significant digits and ordinary rounding approaches.

Notwithstanding that the numerical ranges and parameters setting forth the broad scope are approximations, the numerical values set forth in the specific examples are reported as precisely as possible. Any numerical values, however, inherently contain certain errors necessarily resulting from the standard deviation found in their respective testing measurements.

Protocol for Preparing the Compositions

In the experimental section below, the waxes mentioned are conventional-type waxes, except when it is explicitly indicated that a microwax-type wax is involved.

a. Process for Preparing the Compositions Comprising Only Waxes in the form of MicroParticles

The dyestuffs and also the gelling agent are dispersed, with stirring, in at least one volatile oil, optionally as a mixture with at least one polymer soluble in said oil and having at least one crystallizable portion, and, in such a case, the mixture is preheated to a temperature of 45° C. and then cooled to ambient temperature. The wax in the form of microparticles and also, where appropriate, the remaining ingredients of the composition are then added, with stirring.

The water and/or the at least one water-soluble solvent may be dispersed gradually with stirring.

b. Preparation of the Compositions Comprising both Conventional-Type Waxes and Waxes in the Form of Microparticles

The dyestuffs and also the gelling agent are dispersed, with stirring, in at least one volatile oil, optionally as a mixture with at least one polymer soluble in said volatile oil and having at least one crystallizable portion, and, in such a case, the mixture is preheated to a temperature of 45° C. and then cooled to ambient temperature. The mixture obtained is then heated to 45° C. and the mixture of conventional waxes preheated until they are completely molten is then gradually added. The mixture thus obtained is allowed to return to ambient temperature with stirring. The wax in the form of microparticles and also, where appropriate, the remaining ingredients of the composition are then added.

The water and/or the at least one water-soluble solvent may be dispersed gradually with stirring.

c. Preparation of the Compositions with a Continuous Twin-Screw Blender

The preparation is performed in a continuous twin-screw blender such as the BC-21 model from the company Clextral, and is carried out under the following conditions:
inlet temperature: 10° C.
outlet temperature: 20° C.
flow rate=3 kg/h
screw rate: 600 rpm.

The premolten waxes are introduced at the top of the blender at the same time as the at least one volatile oil and the other ingredients, and the mixture is then cooled, with continuous twin-screw blending, to the outlet temperature.

Measurements of the Physical Characteristics

The solids content is measured according to the protocol described above. The rheology measurements were carried out according to the protocols described above using a Haake RheoStress 600® controlled-stress rheometer, under the following conditions:
measuring temperature 25° C.,
180 sec stage at 25° C. before the start of the measurement,
stress scanning from 1 to 10,000 Pa,
measuring frequency: 1 Hz.

### EXAMPLES 1 TO 3

Three waterproof mascaras were prepared according to the process described in b), said mascaras having, respectively, the compositions given in Table I below (in this table, the amounts indicated are in percentage by weight and are expressed relative to the total weight of the composition):

TABLE I

| | Example 1 | Example 2 | Example 3 |
|---|---|---|---|
| Beeswax | 8.67 | 8.67 | 8.67 |
| Carnauba microwax | 24.2 | 24.2 | 24.2 |
| (MicroCare 350 ® from | | | |
| Micro Powders) | | | |
| Synthetic microwax | 2.02 | 2.02 | 2.02 |
| (MicroEase 114S ® from | | | |
| Micro Powders) | | | |
| Poly(vinyl laurate) | 0.66 | 0.66 | 0.66 |
| (Mexomère PP ® from | | | |
| Chimex) | | | |
| Preserving agent | 0.2 | 0.2 | 0.2 |
| Dye | 5.7 | 5.7 | 5.7 |
| Bentonite | 3.6 | 3.6 | 3.6 |
| Propylene carbonate | 1.18 | 1.18 | 1.18 |
| Allyl stearate/vinyl acetate | 2 | *** | *** |
| copolymer (Mexomère PQ ® | | | |
| from Chimex) | | | |
| Ethylenediamine/stearyl | *** | 2 | *** |
| dilinoleate copolymer | | | |
| (Uniclear 100 VG ® from | | | |
| Arizona Chemical) | | | |
| Glyceryl monostearate | *** | *** | 2 |
| Isododecane | 51.8 | 51.8 | 51.8 |

Various in vitro characteristics of these compositions were studied according to the protocols described above.

The results are given in Table II below:

TABLE II

| | Characteristics | | | |
|---|---|---|---|---|
| Compositions | [Wax] (% m) | DE (% m) | Gp (Pa) | T_{c} (Pa) |
| Example 1 | 34.9 | 48.5 | 4500 | 40 |
| Example 2 | 34.9 | 48.2 | 2300 | 30 |
| Example 3 | 34.9 | 48.5 | 45 000 | 70 |

The compositions obtained comprise more than 20% by weight of wax having a starting melting point of greater than 45° C.

They also have a high solids contents associated with an acceptable plateau modulus of rigidity.

### EXAMPLES 4 AND 5

Two waterproof mascaras having, respectively, the compositions given in Table III below were prepared according to process a) (in this table, the amounts indicated are in percentage by weight and are expressed relative to the total weight of the composition):

TABLE III

| | Example 4 | Example 5 |
|---|---|---|
| Carnauba microwax | 45 | *** |
| (MicroCare 350 ® from Micro Powders) | | |
| Synthetic microwax (MicroEase | *** | 45 |
| 114S ® from Micro Powders) | | |
| Dye | 6 | 6 |
| Bentonite | 3.6 | 3.6 |
| Propylene carbonate | 1.18 | 1.18 |
| Allyl stearate/vinyl acetate copolymer | 6 | 6 |
| (Mexomère PQ ® from CHIMEX) | | |
| Isododecane | 38.22 | 38.22 |

The various characteristics of these compositions were studied in vitro according to the protocols described above.

The results are given in Table IV below:

TABLE IV

| | Characteristics | | | | |
|---|---|---|---|---|---|
| | [Wax] | [Volatile oil] | | | |
| Compositions | (% m) | (% m) | DE (% m) | Gp (Pa) | TC (Pa) |
| Example 4 | 45 | 38.2 | 61.8 | 3 000 | 40 |
| Example 5 | 45 | 38.2 | 61.8 | 160 | 25 |

### EXAMPLES 6 AND 7

Three waterproof mascaras were prepared according to the process described in b) by mixing the ingredients given in Table V below (in this table, the amounts indicated are in percentage by weight and are expressed relative to the total weight of the composition).

TABLE V

| | Example 6 | Example 7 |
|---|---|---|
| Beeswax | 8.67 | 13 |
| Carnauba microwax | 16.2 | 18.37 |
| (MicroCare 350 ® | | |
| from Micro Powders) | | |
| Synthetic microwax | 6.52 | 6.52 |
| (MicroEase 114S ® from | | |
| Micro Powders) | | |
| Poly(vinyl laurate) | 0.66 | *** |
| (Mexomere PP ® from | | |
| Chimex) | | |
| Preserving agent | 0.2 | 0.2 |
| Dye | 5.7 | 5.7 |
| Modified hectorite | 3.6 | 3.6 |
| (Bentone 38V ® from | | |
| Elementis) | | |
| Propylene carbonate | 1.18 | 1.18 |
| Vinyl pyrrolidone/1- | 5 | *** |
| eicosene copolymer | | |
| (Antaron V 220 ® from ISP) | | |
| Allyl stearate/vinyl acetate | 6.5 | 6.5 |
| copolymer | | |
| (Mexomère PQ ® from | | |
| Chimex) | | |
| Ethylenediamine/stearyl | 3 | 1.5 |
| dilinoleate copolymer | | |
| (Uniclear 100 VG ® from | | |
| Arizona Chemical) | | |
| Copolymer of vinyl acetate/ | 3 | *** |
| vinyl t-butyl | | |
| benzoate/crotonic acid in | | |
| aqueous dispersion at | | |
| 26.3% AM | | |
| (Mexomère PAM ® from | | |
| Chimex) | | |
| Copolymer of ethyl | *** | *** |
| acrylate/methyl | | |
| methacrylate (80/20) in | | |
| aqueous dispersion at 50% | | |
| AM | | |
| (Daitosol 5000 AD ® from | | |
| Daito) | | |
| Water | *** | 7.32 |
| Isododecane | 39.77 | 36.11 |

The composition of Example 6 in fact comprises water in a proportion of 2.1% by weight relative to the total weight of the composition, the water originating from the latex used, i.e., the Mexomére PAM® from the company Chimex.

Various in vitro characteristics of these compositions were studied according to the protocols described above.

The results are given in Table VI below.

TABLE VI

| | Characteristics | | |
|---|---|---|---|
| Compositions | DE (% m) | Gp (Pa) | τc (Pa) |
| Example 6 | 57.5 | 7000 | 60 |
| Example 7 | 56.5 | 13 000 | 70 |

### EXAMPLE 8

A waterproof mascara having the following composition was prepared according to the process described in c):

| | | |
|---|---|---|
| Microcrystalline wax (Microwax HW ® from Paramelt) | 30 | g |
| Pigments | 9.24 | g |
| Rice starch | 1.68 | g |
| Vinyl acetate/allyl stearate copolymer | 4.42 | g |
| (Mexomère PQ ® from Chimex) | | |
| Poly(vinyl laurate) (Mexomere PP ® from Chimex) | 1.5 | g |
| Modified hectorite (Bentone 38V ® from Elementis) | 0.63 | g |
| Propylene carbonate | 0.21 | g |
| Ethyl alcohol | 3.0 | g |
| Stearate of 12-hydroxystearic acid oligomer | 0.2 | g |
| (Solsperse 21000 ® from Avecia) | | |
| Preserving agent | 0.4 | g |
| Isododecane | 48.72 | g |

This composition contained 30% by weight of wax having a starting melting point of greater than 45° C. and a solids content of 48.28% by weight relative to the total weight of the composition.

### EXAMPLE 9

A waterproof mascara having the following composition was prepared according to the process described in c):

| | | |
|---|---|---|
| Microcrystalline wax (Microwax HW ® from Paramelt) | 30 | g |
| Modified hectorite Bentone 38V ® from Elementis) | 5 | g |
| Propylene carbonate | 1.7 | g |
| Pigments | 15 | g |
| Ethyl alcohol | 3.0 | g |
| Isododecane | 45.3 | g |

This composition had a solids content of 51.7% by weight relative to the total weight of the composition.

### EXAMPLE 10

A waterproof mascara having the following composition was prepared according to the process described in c):

| | | |
|---|---|---|
| Microcrystalline wax (Microwax HW ® from Paramelt) | 38 | g |
| Vinyl acetate/allyl stearate copolymer | 2.21 | g |
| (Mexomère PQ ® from Chimex) | | |
| Poly(vinyl laurate) (Mexomere PP ® from Chimex) | 0.75 | g |
| Pigments | 4.62 | g |
| Stearate of 12-hydroxystearic acid oligomer | 0.27 | g |
| (Solsperse 21000 ® from Avecia) | | |
| Parleam oil | 10 | g |
| Ethyl alcohol | 3.0 | g |
| Isododecane | 40.11 | g |

This composition contained 30% by weight of a wax having a starting melting point of greater than 45° C. and a solids content of 56.98% by weight relative to the total weight of the composition.

### EXAMPLES 11 TO 15

The waterproof mascaras having the compositions given in Table VII below were prepared according to the process described in c) (in this table, the amounts indicated are in percentage by weight and are expressed relative to the total weight of the composition):

TABLE VII

| | Example | Example | Example | Example | Example |
|---|---|---|---|---|---|
| | 11 | 12 | 13 | 14 | 15 |
| Micro- | 20 | 23 | 25 | 27 | 30 |
| crystalline | | | | | |
| wax | | | | | |
| (Microwax | | | | | |
| HW ® from | | | | | |
| Paramelt) | | | | | |
| Modified | 5 | 5 | 5 | 5 | 5 |
| hectorite | | | | | |
| (Bentone | | | | | |
| 38 V ® | | | | | |
| from | | | | | |
| Elementis) | | | | | |
| Pigments | 15 | 15 | 15 | 15 | 15 |
| Isododecane | 60 | 57 | 55 | 53 | 50 |

The compositions thus obtained make it possible to obtain a make-up which is thickening for the eyelashes and which dries rapidly without decreasing the water resistance thereof.

### EXAMPLE 16

The waterproof mascara having the composition below was prepared according to the process described in c):

| | |
|---|---|
| Carnauba wax | 30 g |
| Modified hectorite (Bentone 38V ® | 5 g |
| from Elementis) | |
| Pigments | 15 g |
| Isododecane | 50 g |

### EXAMPLES 17 TO 21

The waterproof mascaras having the compositions given in Table VIII below were prepared according to the process described in c) (in this table, the amounts indicated are in percentage by weight and are expressed relative to the total weight of the composition).

TABLE VIII

| | Example | Example | Example | Example | Example |
|---|---|---|---|---|---|
| | 17 | 18 | 19 | 20 | 21 |
| Micro- | 10 | 11.5 | 12.5 | 13.5 | 15 |
| crystalline | | | | | |
| wax | | | | | |
| (Microwax | | | | | |
| HW ® from | | | | | |
| Paramelt) | | | | | |
| Carnauba | 10 | 11.5 | 12.5 | 13.5 | 15 |
| wax | | | | | |
| Pigments | 5 | 5 | 5 | 5 | 5 |
| Isododecane | 75 | 72 | 70 | 68 | 65 |

The compositions thus obtained make it possible to obtain a make-up which is thickening for the eyelashes and which dries rapidly without decreasing the water resistance thereof.

### EXAMPLE 22

A W/O foundation having the following composition was prepared:

Phase A

| | | |
|---|---|---|
| Cetyl dimethicone copolyol (Abil EM 90 ® | 3 | g |
| from Goldschmidt) | | |
| Isostearyl diglyceryl succinate (Imwitor 780K ® | 0.6 | g |
| from Condea) | | |
| Mixture of pigments (hydrophobic titanium oxides and | 10 | g |
| iron oxides) | | |
| Carnauba microwax (MicroCare 350 ® from | 22 | g |
| Micro Powders) | | |
| Allyl stearate/vinyl acetate copolymer (Mexomere | 2 | g |
| PQ ® from Chimex) | | |
| Fragrance | 0.5 | g |

| | |
|---|---|
| Isododecane | qs 100 |

Phase B

| | |
|---|---|
| Water | 10 g |
| Magnesium sulphate | 0.7 g |
| Preserving agent (methyl paraben) | 0.2 g |

Phase C

| | |
|---|---|
| Water | 2 g |
| Preserving agent (diazolinyl urea) | 0.25 g |

Phase A was prepared by mixing, with stirring, the cetyl dimethicone copolyol, part of the isododecane, the isostearyl diglyceryl succinate, the wax and the copolymer, and then heating to 50° C. A dispersion, in the other part of the isododecane, of the pigments milled beforehand by means of a three-cylinder mill was first added to this mixture, with stirring, followed by the fragrance.

Phase B was prepared by mixing the various ingredients and then heating to boiling.

The foundation was obtained by emulsifying phase B in phase A with stirring and then adding phase C thereto.

1. A cosmetic composition comprising:
(i) at least 20% by weight of at least one wax having a starting melting point of between 45° C. and 100° C., relative to the total weight of the composition,
(ii) at least one dyestuff, and
(iii) at least one volatile oil.

2. The composition according to claim 1, wherein said composition is a composition for making-up and/or for caring for the skin and/or the lips.

3. The composition according to claim 1, wherein the composition is in a form chosen from a foundation, a blusher, an eye shadow, a lipstick, a care base for the lips, and a care cream for the body and/or the face.

4. The composition according to claim 1, wherein said at least one wax has a melting point of greater than or equal to 50° C.

5. The composition according to claim 4, wherein said at least one wax has a melting point of greater than or equal to 55° C.

6. The composition according to claim 5, wherein said at least one wax has a melting point of greater than or equal to 60° C.

7. The composition according to claim 1, wherein said at least one wax is present in an amount ranging from 20% to 70%, by weight relative to the total weight of the composition.

8. The composition according to claim 7, wherein said at least one wax is present in an amount ranging from 22% to 65% by weight relative to the total weight of the composition.

9. The composition according to claim 8, wherein said at least one wax is present in an amount ranging from 25% to 60% by weight relative to the total weight of the composition.

10. The composition according to claim 1, wherein said at least one wax is chosen from carnauba wax, rice bran wax, candelilla wax, ouricury wax, montan wax, ozokerite, waxes obtained by Fisher-Tropsch synthesis, hydrogenated jojoba oil, bis(1,1,1-trimethylol-propane)tetrabehenate, waxes obtained by catalytic hydrogenation of olive oil esterified with stearyl alcohol, microcrystalline waxes, and polyethylene waxes.

11. The composition according to claim 1, further comprising at least one second wax having a starting melting point of less than or equal to 45° C.

12. The composition according to claim 11, wherein the at least one second wax is chosen from hydrocarbon-based waxes, waxes obtained by catalytic hydrogenation of animal or plant oils containing linear or branched C₈-C₃₂ fatty chains, and waxes obtained by hydrogenation of castor oil esterified with cetyl alcohol.

13. The composition according to claim 12, wherein said hydrocarbon-based waxes are chosen from beeswax, lanolin wax, China insect waxes, sumac wax, paraffins, polyethylene waxes, waxy copolymers, and esters thereof.

14. The composition according to claim 12, wherein the waxes obtained by catalytic hydrogenation of animal or plant oils containing linear or branched C₈-C₃₂ fatty chains are chosen from trans-isomerized partially hydrogenated jojoba oil, hydrogenated sunflower oil, hydrogenated castor oil, hydrogenated coconut oil, hydrogenated lanolin oil, and bis(1,1,1-trimethylolpropane)-tetrastearate.

15. The composition according to claim 11, wherein the at least one second wax is chosen from waxes having a tack of greater than or equal to 0.7 N.s, and a hardness of less than or equal to 3.5 MPa.

16. The composition according to claim 15, wherein said at least one second wax is chosen from C₂₀-C₄₀ alkyl(hydroxystearyloxy)stearates.

17. The composition according to claim 11, wherein the at least one second wax having a starting melting point of less than or equal to 45° C. is present in an amount ranging from 0.1% to 50%, by weight relative to the total weight of the composition.

18. The composition according to claim 17, wherein the at least one second wax having a starting melting point of less or equal to than 45° C. is present in an amount ranging from 1% to 45% by weight relative to the total weight of the composition.

19. The composition according to claim 18, wherein at least one second wax having a starting melting point of less than or equal to 45° C. is present in an amount ranging from 5% to 40% by weight relative to the total weight of the composition.

20. The composition according to claim 1, wherein said at least one volatile oil is chosen from hydrocarbon-based oils, silicone oils, and fluoro oils.

21. The composition according to claim 20, wherein said at least one hydrocarbon-based volatile oil is chosen from hydrocarbon-based oils comprising from 8 to 16 carbon atoms.

22. The composition according to claim 21, wherein the hydrocarbon-based oils comprising from 8 to 16 carbon atoms are chosen from branched C₈-C₁₆ alkanes, branched C₈-C₁₆ esters, and petroleum distillates.

23. The composition according to claim 22, wherein the branched C₈-C₁₆ alkanes are chosen from petroleum-based C₈-C₁₆ isoalkanes.

24. The composition according to claim 23, wherein the C₈-C₁₆ isoalkanes are chosen from isododecane, isodecane, and isohexadecane.

25. The composition according to claim 22, wherein the branched C₈-C₁₆ esters are isohexyl neopentanoate.

26. The composition according to claim 1, wherein said at least one volatile oil is present in an amount of between 5 and 80% by weight, relative to the total weight of the composition.

27. The composition according to claim 26, wherein the at least one volatile oil is present in an amount ranging from 15 to 75% by weight, relative to the total weight of the composition.

28. The composition according to claim 1, wherein said at least one volatile oil is present in an amount of less than 70% by weight, relative to the total weight of the composition.

29. The composition according to claim 28, wherein the at least one volatile oil is present in an amount ranging from 20 to 65% by weight, relative to the total weight of the composition.

30. The composition according to claim 1, further comprising at least one non-volatile oil.

31. The composition according to claim 1, further comprising water and/or at least one water-soluble solvent present in an amount of less than or equal to 20%, by weight relative to the total weight of the composition.

32. The composition according to claim 31, wherein the water and/or at least one water-soluble solvent is present in an amount ranging from 1 to 18% by weight relative to the total weight of the composition.

33. The composition according to claim 32, wherein the water and/or at least one water-soluble solvent are present in an amount ranging from 2 to 15% by weight relative to the total weight of the composition.

34. The composition according to claim 31, wherein said at least one water-soluble solvent is chosen from lower monoalcohols comprising from 1 to 5 carbon atoms, glycols comprising from 2 to 8 carbon atoms, C₃ and C₄ ketones, and C₂ to C₄ aldehydes.

35. The composition according to claim 1, wherein said composition is free of water and of water-soluble solvent.

36. The composition according to claim 1, further comprising at least one polymer soluble in said at least one volatile oil and having at least one crystallizable portion.

37. The composition according to claim 36, wherein said at least one polymer has a molar mass ranging from 200 to 1,000,000 g/mol.

38. The composition according to claim 36, wherein said crystallizable portion is present in an amount ranging from 5% to 50%, by weight of the total weight of said at least one polymer.

39. The composition according to claim 36, wherein said at least one polymer is chosen from copolymers of linear and saturated C₁₂ to C₃₀ alkyl(meth)acrylates and of linear C₄ to C₁₀ or branched or cyclic and/or unsaturated C₄ to C₃₀ alkyl (meth)acrylates, copolymers of vinyl esters comprising linear and saturated C₁₂ to C₃₀ alkyl groups and of vinyl esters containing linear C₄ to C₁₀ or branched or cyclic and/or unsaturated C₄ to C₃₀ alkyl groups, polyamide polycondensates resulting from condensation between (a) at least one acid chosen from dicarboxylic acids comprising at least 32 carbon atoms and (b) an alkylenediamine, said polycondensate comprising at least one end carboxylic acid group esterified or amidated with at least one monoalcohol or one monoamine comprising from 12 to 30 linear and saturated carbon atoms; and lipophilic polyester polycondensates in which the ends are esterified with a crystallizable acid or alcohol comprising a saturated, linear C₁₂ to C₃₀ carbon chain.

40. The composition according to claim 36, wherein said at least one polymer is chosen from the copolymers vinyl acetate/vinyl stearate, vinyl acetate/allyl stearate, vinyl acetate/ethylene, and ethylenediamine/stearyl dilinoleate; and the block copolymers hydrogenated butadiene/isoprene and 12-polyhydroxystearic acid in which at least one of the ends is esterified with stearic acid.

41. The composition according to claim 36, wherein said at least one polymer is present in an amount ranging from 0.01 to 30%, by weight relative to the total weight of the composition.

42. The composition according to claim 1, further comprising at least one film-forming polymer.

43. The composition according to claim 1, wherein the composition further comprises at least one filler.

44. The composition according to claim 1, wherein the composition further comprises at least one cosmetically acceptable additive chosen from at least one of antioxidants, preserving agents, fragrances, neutralizing agents, plasticizers, fibers, gelling agents, and cosmetic active agents.

45. A process for preparing a composition comprising:
(i) at least 20% by weight of at least one wax having a starting melting point of greater than 45° C., relative to the total weight of the composition,
(ii) at least one dyestuff, and
(iii) at least one volatile oil,
said process comprising
continuously blending the at least one wax having a starting melting point of greater than 45° C.; and
continuously cooling the at least one wax from a temperature above the melting temperature of said wax to ambient temperature.

46. The process according to claim 45, wherein said blending is carried out by a continuous twin-screw blender.

47. The process according to claim 45, wherein said at least one wax has a melting point of greater than or equal to 50° C.

48. The process according to claim 47, wherein said at least one wax has a melting point of greater than or equal to 55° C.

49. The process according to claim 48, wherein said at least one wax has a melting point of greater than or equal to 60° C.

50. The process according to claim 45, wherein said at least one wax is present in an amount ranging from 20% to 70%, by weight relative to the total weight of the composition.

51. The process according to claim 50, wherein said at least one wax is present in an amount ranging from 22% to 65% by weight relative to the total weight of the composition.

52. The process according to claim 51, wherein said at least one wax is present in an amount ranging from 25% to 60% by weight relative to the total weight of the composition.

53. The process according to claim 45, wherein said at least one wax is chosen from carnauba wax, rice bran wax, candelilla wax, ouricury wax, montan wax, ozokerite, waxes obtained by Fisher-Tropsch synthesis, hydrogenated jojoba oil, bis(1,1,1-trimethylolpropane)tetrabehenate, waxes obtained by catalytic hydrogenation of olive oil esterified with stearyl alcohol, microcrystalline waxes, and polyethylene waxes.

54. The process according to claim 45, wherein at least one volatile oil is added either prior to said blending or in the course of said blending.

55. A process for preparing a composition comprising,
(i) at least 20% by weight of at least one wax having a starting melting point greater than 45° C., relative to the total weight of the composition,
(ii) at least one dyestuff, and
(iii) at least one volatile oil,
said process comprising
dispersing the at least one wax in the form of particles having a size ranging from 0.5 μm to 30 μm and having a starting melting point of greater than 45° C., in at least one volatile oil, said at least one oil being at a temperature below the melting temperature of said at least one wax in the form of particles.

56. The process according to claim 55, wherein said dispersing is carried out at ambient temperature.

57. The process according to claim 55, wherein said particles have a size ranging from 1 to 20 μm.

58. The process according to claim 55, wherein said at least one wax is chosen from at least one of carnauba waxes and synthetic waxes.

59. The process according to claim 55, wherein said at least one wax is in molten form and is introduced beforehand into said at least one volatile oil, and then the mixture thus obtained is allowed to cool, with stirring or blending, to a temperature at least below the melting temperature of said wax in the form of particles.

60. The process according to claim 55, wherein said at least one volatile oil is chosen from hydrocarbon-based oils, silicone oils, and fluoro oils.

61. The process according to claim 60, wherein said at least one hydrocarbon-based volatile oil is chosen from hydrocarbon-based oils comprising from 8 to 16 carbon atoms.

62. The process according to claim 61, wherein the hydrocarbon-based oils comprising from 8 to 16 carbon atoms are chosen from branched C₈-C₁₆ alkanes, branched C₈-C₁₆ esters, and petroleum distillates.

63. The process according to claim 62, wherein the branched C₈-C₁₆ alkanes are chosen from petroleum-based C₈-C₁₆ isoalkanes.

64. The process according to claim 63, wherein the C₈-C₁₆ isoalkanes are chosen from isododecane, isodecane, and isohexadecane.

65. The process according to claim 62, wherein the branched C₈-C₁₆ esters are isohexyl neopentanoate.

66. The process according to claim 55, wherein said at least one volatile oil is present in an amount of between 5 and 80% by weight, relative to the total weight of the composition.

67. The process according to claim 66, wherein the at least one volatile oil is present in an amount ranging from 15 to 75% by weight, relative to the total weight of the composition.

68. The process according to claim 55, wherein said at least one volatile oil is present in an amount of less than 70% by weight, relative to the total weight of the composition.

69. The process according to claim 68, wherein the at least one volatile oil is present in an amount ranging from 20 to 65% by weight, relative to the total weight of the composition.

70. A cosmetic method for caring for and/or for making-up the skin and/or the lips, comprising applying to the skin and/or the lips a composition comprising
(i) at least 20% by weight of at least one wax having a starting melting point of between 45° C. and 100° C., relative to the total weight of the composition,
(ii) at least one dyestuff, and
(iii) at least one volatile oil.

71. A cosmetic method for making-up keratin fibers comprising applying to said keratin fibers a composition comprising:
(i) no more than 20% by weight of water and/or of at least one water-soluble solvent,
(ii) at least 20% by weight of at least one wax having a starting melting point greater than 45° C., relative to the total weight of the composition,
(iii) at least one dyestuff, and
(iv) at least one volatile oil.

72. The method according to claim 71, wherein said keratin fibers are eyelashes.

73. A cosmetic method for making-up keratin fibers, comprising applying to keratin fibers a composition comprising
(i) no more than 20% by weight of water and/or of at least one water-soluble solvent,
(ii) at least 20% by weight of at least one wax having a starting melting point greater than 45° C., relative to the total weight of the composition, and
(iii) at least one volatile oil.

74. The method according to claim 73, wherein said keratin fibers are eyelashes.

75. A make-up composition for keratin fibers comprising:
(i) 20% or less by weight of water and/or of at least one water-soluble solvent,
(ii) at least 20% by weight of at least one wax having a starting melting point of greater than 45° C., relative to the total weight of the composition,
(iii) at least one dyestuff, and
(iv) at least one volatile oil.

76. The composition according to claim 75, wherein said composition is a composition for making-up and/or for caring for the skin and/or the lips.

77. The composition according to claim 75, wherein the composition is in a form chosen from a foundation, a blusher, an eye shadow, a lipstick, a care base for the lips, and a care cream for the body and/or the face.

78. The composition according to claim 75, wherein said at least one wax has a melting point of greater than or equal to 50° C.

79. The composition according to claim 78, wherein said at least one wax has a melting point of greater than or equal to 55° C.

80. The composition according to claim 79, wherein said at least one wax has a melting point of greater than or equal to 60° C.

81. The composition according to claim 75, wherein said at least one wax is present in an amount ranging from 20% to 70%, by weight relative to the total weight of the composition.

82. The composition according to claim 81, wherein said at least one wax is present in an amount ranging from 22% to 65% by weight relative to the total weight of the composition.

83. The composition according to claim 82, wherein said at least one wax is present in an amount ranging from 25% to 60% by weight relative to the total weight of the composition.

84. The composition according to claim 75, wherein said at least one wax is chosen from carnauba wax, rice bran wax, candelilla wax, ouricury wax, montan wax, ozokerite, waxes obtained by Fisher-Tropsch synthesis, hydrogenated jojoba oil, bis(1,1,1-trimethylol-propane)tetrabehenate, waxes obtained by catalytic hydrogenation of olive oil esterified with stearyl alcohol, microcrystalline waxes, and polyethylene waxes.

85. The composition according to claim 75, further comprising at least one second wax having a starting melting point of less than or equal to 45° C.

86. The composition according to claim 85, wherein the at least one second wax is chosen from hydrocarbon-based waxes, waxes obtained by catalytic hydrogenation of animal or plant oils containing linear or branched C₈-C₃₂ fatty chains, and waxes obtained by hydrogenation of castor oil esterified with cetyl alcohol.

87. The composition according to claim 86, wherein said hydrocarbon-based waxes are chosen from beeswax, lanolin wax, China insect waxes, sumac wax, paraffins, polyethylene waxes, waxy copolymers, and esters thereof.

88. The composition according to claim 86, wherein the waxes obtained by catalytic hydrogenation of animal or plant oils containing linear or branched C₈-C₃₂ fatty chains are chosen from trans-isomerized partially hydrogenated jojoba oil, hydrogenated sunflower oil, hydrogenated castor oil, hydrogenated coconut oil, hydrogenated lanolin oil, and bis(1,1,1-trimethylolpropane)-tetrastearate.

89. The composition according to claim 85, wherein the at least one second wax is chosen from waxes having a tack of greater than or equal to 0.7 N.s, and a hardness of less than or equal to 3.5 MPa.

90. The composition according to claim 89, wherein said at least one second wax is chosen from C₂₀-C₄₀ alkyl(hydroxystearyloxy)stearates.

91. The composition according to claim 85, wherein the at least one second wax having a starting melting point of less than or equal to 45° C. is present in an amount ranging from 0.1% to 50%, by weight relative to the total weight of the composition.

92. The composition according to claim 91, wherein the at least one second wax having a starting melting point of less or equal to than 45° C. is present in an amount ranging from 1% to 45% by weight relative to the total weight of the composition.

93. The composition according to claim 92, wherein at least one second wax having a starting melting point of less than or equal to 45° C. is present in an amount ranging from 5% to 40% by weight relative to the total weight of the composition.

94. The composition according to claim 75, wherein said at least one volatile oil is chosen from hydrocarbon-based oils, silicone oils, and fluoro oils.

95. The composition according to claim 94, wherein said at least one hydrocarbon-based volatile oil is chosen from hydrocarbon-based oils comprising from 8 to 16 carbon atoms.

96. The composition according to claim 95, wherein the hydrocarbon-based oils comprising from 8 to 16 carbon atoms are chosen from branched C₈-C₁₆ alkanes, branched C₈-C₁₆ esters, and petroleum distillates.

97. The composition according to claim 96, wherein the branched C₈-C₁₆ alkanes are chosen from petroleum-based C₈-C₁₆ isoalkanes.

98. The composition according to claim 97, wherein the C₈-C₁₆ isoalkanes are chosen from isododecane, isodecane, and isohexadecane.

99. The composition according to claim 96, wherein the branched C₈-C₁₆ esters are isohexyl neopentanoate.

100. The composition according to claim 75, wherein said at least one volatile oil is present in an amount of between 5 and 80% by weight, relative to the total weight of the composition.

101. The composition according to claim 100, wherein the at least one volatile oil is present in an amount ranging from 15 to 75% by weight, relative to the total weight of the composition.

102. The composition according to claim 75, wherein said at least one volatile oil is present in an amount of less than 70% by weight, relative to the total weight of the composition.

103. The composition according to claim 102, wherein the at least one volatile oil is present in an amount ranging from 20 to 65% by weight, relative to the total weight of the composition.

104. The composition according to claim 75, further comprising at least one non-volatile oil.

105. The composition according to claim 75, further comprising water and/or at least one water-soluble solvent present in an amount of less than or equal to 20%, by weight relative to the total weight of the composition.

106. The composition according to claim 105, wherein the water and/or at least one water-soluble solvent is present in an amount ranging from 1 to 18% by weight relative to the total weight of the composition.

107. The composition according to claim 106, wherein the water and/or at least one water-soluble solvent are present in an amount ranging from 2 to 15% by weight relative to the total weight of the composition.

108. The composition according to claim 105, wherein said at least one water-soluble solvent is chosen from lower monoalcohols comprising from 1 to 5 carbon atoms, glycols comprising from 2 to 8 carbon atoms, C₃ and C₄ ketones, and C₂ to C₄ aldehydes.

109. The composition according to claim 75, wherein said composition is free of water and of water-soluble solvent.

110. The composition according to claim 75, further comprising at least one polymer soluble in said at least one volatile oil and having at least one crystallizable portion.

111. The composition according to claim 110, wherein said at least one polymer has a molar mass ranging from 200 to 1,000,000 g/mol.

112. The composition according to claim 110, wherein said crystallizable portion is present in an amount ranging from 5% to 50%, by weight of the total weight of said at least one polymer.

113. The composition according to claim 110, wherein said at least one polymer is chosen from copolymers of linear and saturated C₁₂ to C₃₀ alkyl(meth)acrylates and of linear C₄ to C₁₀ or branched or cyclic and/or unsaturated C₄ to C₃₋₀ alkyl(meth)acrylates, copolymers of vinyl esters comprising linear and saturated C₁₂ to C₃₀ alkyl groups and of vinyl esters containing linear C₄ to C₁₀ or branched or cyclic and/or unsaturated C₄ to C₃₀ alkyl groups, polyamide polycondensates resulting from condensation between (a) at least one acid chosen from dicarboxylic acids comprising at least 32 carbon atoms and (b) an alkylenediamine, said polycondensate comprising at least one end carboxylic acid group esterified or amidated with at least one monoalcohol or one monoamine comprising from 12 to 30 linear and saturated carbon atoms; and lipophilic polyester polycondensates in which the ends are esterified with a crystallizable acid or alcohol comprising a saturated, linear C₁₂ to C₃₀ carbon chain.

114. The composition according to claim 110, wherein said at least one polymer is chosen from the copolymers vinyl acetate/vinyl stearate, vinyl acetate/allyl stearate, vinyl acetate/ethylene, and ethylenediamine/stearyl dilinoleate; and the block copolymers hydrogenated butadiene/isoprene and 12-polyhydroxystearic acid in which at least one of the ends is esterified with stearic acid.

115. The composition according to claim 110, wherein said at least one polymer is present in an amount ranging from 0.01 to 30%, by weight relative to the total weight of the composition.

116. The composition according to claim 75, further comprising at least one film-forming polymer.

117. The composition according to claim 75, wherein the composition further comprises at least one filler.

118. The composition according to claim 75, wherein the composition further comprises at least one cosmetically acceptable additive chosen from at least one of antioxidants, preserving agents, fragrances, neutralizing agents, plasticizers, fibers, gelling agents, and cosmetic active agents.
